(19) United States
(12) Patent Application Publication
(10) Pub. No.: US 2006/0211739 A1
(43) Pub. Date: Sep. 21, 2006

(54) Use of selective P2X7 receptor antagonists

(76) Inventors: Arturo Perez-Medrano, Grayslake, IL (US); Derek W. Nelson, Highland Park, IL (US); William A. Carroll, Evanston, IL (US); Michael E. Kort, Lake Bluff, IL (US); Robert J. Gregg, Libertyville, IL (US); Eric A. Voight, Downingtown, PA (US); Michael F. Jarvis, Vernon Hills, IL (US); Elizabeth A. Kowaluk, Highland Park, IL (US)

Correspondence Address: Robert DeBerardine;D-377/AP6A-1, Abbott Laboratories, 100 Abbott Park Road, Abbott Park, IL, 60064-6008, US

(21) Appl. No.: 11/344,920

(22) Filed: Feb. 1, 2006

Related U.S. Application Data
(60) Provisional application No. 60/650,835, filed on Feb. 8, 2005.

(51) Int. Cl. A61K 31/4439; A61K 31/41

(52) U.S. Cl. 514/341; 514/381

(57) ABSTRACT

The present invention relates to the use of selective P2X₇ receptor antagonists of formula I, or a pharmaceutically acceptable salt or prodrug thereof 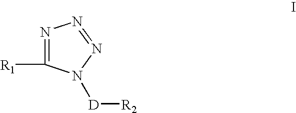
wherein D, R₁ and R₂ are as defined in claim 1, for the treatment of neuropathic pain, chronic inflammatory pain, inflammation, neurodegeneration and for promoting neuroregeneration,

This application claims priority to the provisional application Ser. No. 60/650,835 filed on Feb. 8, 2005.

### TECHNICAL FIELD

The present invention relates to the use of selective P2X₇ receptor antagonists and to compositions containing selective P2X₇ receptor antagonists for the treatment of neuropathic pain, chronic inflammatory pain, inflammation, neurodegeneration and, for promoting neuroregeneration.

### BACKGROUND OF THE INVENTION

P2X receptors are ionotropic receptors activated by ATP. The importance of P2X receptors in nociception is underscored by the variety of pain states in which this endogenous ligand can be released. Of the seven P2X receptors, the P2X₇ is distinguished by its ability to form a large pore upon prolonged or repeated agonist stimulation. It is partially activated by saturating concentrations of ATP, whereas it is fully activated by the synthetic ATP analog benzoylbenzoic ATP (BzATP) (Bianchi et al., Eur. J. Pharmacol, Vol. 376, pages 127-138, 1999). The P2X₇ receptor is expressed by presynaptic terminals in the central and peripheral nervous systems, antigen-presenting cells including macrophages, human epidermal Langerhans' cells, microglial cells and a number of tumor cell lines of varying origin (Jacobson K A, et al. “Adenosine and Adenine Nucleotides: From Molecular Biology to Integrative Physiology”. L. Belardinelli and A. Pelleg (eds.), Kluwer, Boston, pages 149-166, 1995).

Recent studies demonstrated the participation of P2X₇ receptors in the modulation of electrical stimulation and ATP-evoked GABA and glutamate release from mouse hippocampal slices (Papp et al., Neuropharmacology and Neurotoxicology Vol. 15, pages 2387-2391, 2004)). In the central nervous system, the P2X₇ receptor is predominately expressed by microglia, the resident macrophages of the brain. On glial cells, the P2X₇ receptor has been shown to mediate release of glutamate (Anderson C. et al. Drug Dev. Res, Vol. 50. page 92, 2000). Upregulation of the P2X₇ receptor, most likely on activated microglia, was reported in association with ischemic damage and necrosis induced by occlusion of middle cerebral artery in rat brain (Collo G. et al. Neuropharmacology, Vol. 36, pages 1277-1283, 1997).

Recent studies indicate a role of the P2X₇ receptor in the generation of superoxide in microglia, and upregulation of P2X₇ receptors around β-amyloid plaques in a transgenic mouse model for Alzheimer's disease (Parvathenani et al., J. Biol. Chemistry, Vol. 278, pages 13300-13317, 2003) and in multiple sclerosis lesions from autopsy brain sections (Narcisse et al., Glia, Vol. 49, pages 245-258 (2005). Activation of the P2X₇ receptor on cells of the immune system (macrophages, mast cells and lymphocytes) leads to release of interleukin-1β (IL-1β), giant cell formation, degranulation, and L-selectin shedding. ATP has been shown to increase local release and process of IL-1β following lipopolysaccharide S (LPS) intraperitoneal injections in rats through a X₇ receptor mediated mechanism (Griffiths et al., J. Immunology Vol. 154, pages 2821-2828 (1995); Solle et al., J. Biol. Chemistry, Vol. 276, pages 125-132, (2001)).

Oxidized ATP (oATP), a nonselective and irreversible P2X₇ antagonist, was recently reported to possess peripherally mediated antinociceptive properties in inflamed rats (Dell'Antonio et al. Neuroscience Lett, Vol. 327, pages 87-90, 2002). Activation of P2X₇ receptors localized on presynaptic terminals in the central and peripheral nervous systems (Deuchars et al J. Neuroscience, Vol. 21, pages 7143-7152, 2001) induced release of the excitatory amino acid neurotransmitter glutamate. A link between a P2X₇ purinoceptor gene and chronic, inflammatory and neuropathic pain has also been reported (Hatcher et al., The 6th International Conference on the Mechanisms and Treatment of Neuropathic Pain. San Fransisco, Calif.—Sep. 18-20, 2003).

Antagonists to the P2X₇ receptor significantly improved functional recovery and decreased cell death in spinal cord injury (SCI) animal models. Rats with SCI were administered P2X₇ receptor irreversible antagonists oATP and PPADS with a resulting decrease of histological injury and improved recovery of motor function after the lesions (Wang et al., Nature Medicine Vol. 10, pages B21-B27, 2004).

Taken together, these findings indicate that compounds acting at the P2X₇ receptor may have utility in the treatment of pain, inflammatory processes, and degenerative conditions associated with disease states such as rheumatoid arthritis, osteoarthritis, psoriasis, allergic dermatitis, asthma, chronic obstructive pulmonary disease, airways hyper-responsiveness, septic shock, glomerulonephritis, irritable bowel disease, Crohn's disease, ulcerative colitis, atherosclerosis, growth and metastases of malignant cells, myoblastic leukaemia, diabetes, Alzheimer's disease, multiple sclerosis, meningitis, osteoporosis, burn injury, ischemic heart disease, stroke and varicose veins.

In view of the above facts, there is a need for selective P2X₇ antagonist that can be efficiently used in preventing, treating, or ameliorating states as neuropathic pain, chronic inflammatory pain, inflammation and neurodegenerative conditions associated with several progressive CNS disorders, including, but not limited to, Alzheimer's disease, Parkinson's disease, amyotrophic lateral sclerosis, Huntington's disease, dementia with Lewy bodies, multiple sclerosis as well as diminished CNS function resulting from traumatic brain injury.

### SUMMARY OF THE INVENTION

In its principal embodiment, the invention relates to a method of treating neuropathic pain, chronic inflammatory pain, inflammation, neurodegeneration and of promoting neruoregeneration comprising administering a therapeutically effective amount of a selective P2X₇ receptor antagonist of formula I 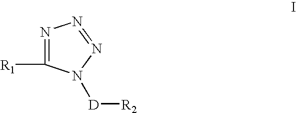
or a pharmaceutically acceptable salt, prodrug, or salt of a prodrug thereof, wherein

D is a bond or C₁₋₅ alkylene;

R₁ is selected form the groups consisting of cycloalkyl, cycloalkenyl, heterocycle, aryl, heteroaryl, arylalkyl and heteroarylalkyl; wherein each R₁ is substituted with 0, 1, 2, 3, 4 or 5 substituents independently selected from the group consisting of alkyl, alkenyl, alkynyl, nitro, cyano, halo, —OR_{c}, —O(CO)R_{c}, —OC(O)OR_{c}, —OS(O)₂R_{c}, —SR_{c}, —S(O)R_{c}, —S(O)₂R_{c}, —S(O)₂OR_{c}, —S(O)₂NR_{c}R_{d}, —NR_{c}R_{d}, —N(R_{d})C(O)OR_{c}, —N(R_{d})C(O)NR_{c}R_{d}, —N(R_{d})S(O)₂NR_{c}R_{d}, —C(O)R_{c}, —C(O)OR_{c}, —C(O)NR_{c}R_{d}, haloalkyl, cyanoalkyl, nitroalkyl, -alkylOR_{c}, —O(CO)R_{c}, -alkylOC(O)OR_{c}, -alkylOS(O)₂R_{c}, -alkylSR_{c}, -alkylS(O)R_{c}, -alkylS(O)₂R_{c}, -alkylS(O)₂OR_{c}, -alkylS(O)₂NR_{c}R_{d}, -alkylNR_{c}R_{d}, -alkylN(R_{d})C(O)OR_{c}, -alkylN(R_{d})C(O)NR_{c}R_{d}, -alkylN(R_{d})S(O)₂NR_{c}R_{d}, -alkylC(O)R_{c}, -alkylC(O)OR_{c}, -alkylC(O)NR_{c}R_{d} and R₃; provided that when R₁ is arylalkyl or heteroarylalkyl, D is a bond;

R₂ is selected form the groups consisting of cycloalkyl, cycloalkenyl, heterocycle, aryl, and heteroaryl; wherein each R₂ is substituted with 0, 1, 2, 3, 4 or 5 substituents independently selected from the group consisting of alkyl, alkenyl, alkynyl, nitro, cyano, halo, —OR_{c}, —O(CO)R_{c}, —OC(O)OR_{c}, —OS(O)₂R_{c}, —SR_{c}, —S(O)R_{c}, —S(O)₂R_{c}, —S(O)₂OR_{c}, —S(O)₂NR_{c}R_{d}, —NR_{c}R_{d}, —N(R_{d})C(O)OR_{c}, —N(R_{d})C(O)NR_{c}R_{d}, —N(R_{d})S(O)₂NR_{c}R_{d}, —C(O)R_{c}, —C(O)OR_{c}, —C(O)NR_{c}R_{d}, haloalkyl, cyanoalkyl, nitroalkyl, -alkylOR_{c}, —O(CO)R_{c}, -alkylOC(O)OR_{c}, -alkylOS(O)₂R_{c}, -alkylSR_{c}, -alkylS(O)R_{c}, -alkylS(O)₂R_{c}, -alkylS(O)₂OR_{c}, -alkylS(O)₂NR_{c}R_{d}, -alkylNR_{c}R_{d}, -alkylN(R_{d})C(O)OR_{c}, -alkylN(R_{d})C(O)NR_{c}R_{d}, -alkylN(R_{d})S(O)₂NR_{c}R_{d}, -alkylC(O)R_{c}, -alkylC(O)OR_{c}, -alkylC(O)NR_{c}R_{d} and R₃;

R₃ is selected from the group consisting of cycloalkyl, cycloalkenyl, heterocycle, aryl, and heteroaryl; wherein each R₃ is independently substituted with 0, 1, 2, 3, 4 or 5 substituents independently selected from the group consisting of alkyl, alkenyl, alkynyl, nitro, cyano, halo, formyl, hydroxy, alkoxy, haloalkoxy, —OC(O)alkyl, —S(O)₂alkyl, —S(O)₂NH₂, —S(O)₂N(H)(alkyl), —S(O)₂N(alkyl)₂, —NH₂, —N(H)(alkyl), —N(alkyl)₂, —C(O)(alkyl), —C(O)(OH), —C(O)(Oalkyl), —C(O)NH₂, —C(O)N(H)(alkyl), —C(O)N(alkyl)₂, haloalkyl, formylalkyl, cyanoalkyl, nitroalkyl, hydroxyalkyl, alkoxyalkyl, haloalkoxyalkyl, -alkylOC(O)alkyl, -alkyl-S(O)₂alkyl, -alkyl-S(O)₂NH₂, -alkyl-S(O)₂N(H)(alkyl), -alkyl-S(O)₂N(alkyl)₂, -alkyl-NH₂, -alkyl-N(H)(alkyl), -alkyl-N(alkyl)₂, -alkyl-C(O)(alkyl), -alkyl-C(O)(OH), -alkyl-C(O)(Oalkyl), -alkyl-C(O)NH₂, -alkyl-C(O)N(H)(alkyl), and -alkyl-C(O)N(alkyl)₂; and

R_{c} and R_{d}, at each occurrence, are independently selected from the group consisting of hydrogen, alkyl, haloalkyl, aryl, heteroaryl, arylalkyl and heteroarylalkyl; wherein the aryl, heteroaryl, aryl moiety of the arylalkyl and heteroaryl moiety of the heteroarylalkyl are independently substituted with 0, 1, 2 or 3 substituents independently selected from the group consisting of alkyl, halo, haloalkyl, hydroxyl, hydroxyalkyl and alkoxyalky.

### DETAILED DESCRIPTION OF THE INVENTION

All references contained herein are fully incorporated by reference.

Definition of Terms

The term “alkenyl” as used herein, refers to a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “C₁₋₅ alkylene” as used herein, means a divalent group derived from a straight or branched chain hydrocarbon of from 1 to 5 carbon atoms. Representative examples of C₁₋₅ alkylene include, but are not limited to, —CH₂—, —CH(CH₃)—, —C(CH₃)₂—, —CH₂CH₂—, —CH₂CH₂CH₂—, —CH₂CH₂CH₂CH₂—, —CH₂CH₂CH₂CH₂CH₂— and —CH₂CH(CH₃)CH₂—.

The term “alkynyl” as used herein, refers to a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl” as used herein, means a phenyl group, or a bicyclic or a tricyclic hydrocarbon fused ring system containing zero heteroatom wherein one or more of the fused rings is a phenyl group. Bicyclic hydrocarbon fused ring systems are exemplified by a phenyl group fused to a monocyclic cycloalkyl group, as defined herein, a monocyclic cycloalkenyl group, as defined herein, or another phenyl group. Tricyclic hydrocarbon fused ring systems are exemplified by the bicyclic fused hydrocarbon ring system as defined hereinabove, fused to a monocyclic cycloalkyl group, as defined herein, a monocyclic cycloalkenyl group, as defined herein, or another phenyl group. The aryl groups of the present invention are appended to the parent moiety through any substitutable atoms in the group. The aryl groups of the present invention can be unsubstituted or substituted. Representative examples of aryl include, but are not limited to, 9,10-dihydro-anthracen-9-yl, fluorenyl, 2,3-dihydro-1H-inden-1-yl, indan-4-yl, indan-5-yl, inden-1-yl, naphthyl, phenyl, 1,2,3,4-tetrahydronaphthalen-2-yl and tetrahydronaphthalenyl.

The term “arylalkyl” as used herein, refers to an aryl group, as used herein, appended to the parent moiety through an alkyl group as defined herein.

The term “cyano” as used herein, refers to —CN.

The term “cyanoalkyl” as used herein, refers to an alkyl group as defined herein, in which one or two hydrogen atoms are replaced by cyano. Representative examples of cyanoalkyl include, but are not limited to, 1-methyl-1-cyanoethyl and cyanoethyl.

The term “cycloalkyl” or “cycloalkane” as used herein, refers to a saturated monocyclic hydrocarbon ring system having three to eight carbon atoms and zero heteroatom. Examples of monocyclic ring systems include cyclopropyl, cyclobutyl, cyclopentyl, cyclohexyl, cycloheptyl, and cyclooctyl. The term “cycloalkyl” of the present invention also include a bicyclic fused ring system wherein the monocyclic cycloalkyl ring is fused to another monocyclic cycloalkyl group, as defined herein The cycloalkyl groups of the present invention can be unsubstituted or substituted, and are connected to the parent molecula moiety through any substitutable carbon atom of the group.

The term “cycloalkenyl” or “cycloalkene” as used herein, refers to a non-aromatic, partially unsaturated, monocyclic hydrocarbon ring system, having 4, 5, 6, 7 or 8 carbon atoms and zero heteroatom. The 4-membered ring systems have one double bond, the 5- or 6-membered ring systems have one or two double bonds, and the 7- or 8-membered ring systems have one, two or three double bonds. Representative examples of cycloalkenyl groups include, but not limited to, cyclobutenyl, cyclopentenyl, cyclohexenyl, cycloheptenyl and cyclooctenyl. The term “cycloalkenyl” of the present invention also include a bicyclic fused ring system wherein the monocyclic cycloalkenyl ring is fused to a monocyclic cycloalkyl group, as defined herein, or another monocyclic cycloalkenyl group, as defined herein. Representative examples of the bicyclic cycloalkenyl groups include, but not limited to, 4,5,6,7-tetrahydro-3aH-indene, octahydronaphthalenyl and 1,6-dihydro-pentalene. The cycloalkenyl groups of the present invention can be unsubstituted or substituted, and are attached to the parent molecular moiety through any substitutable carbon atom of the group.

The term “formyl” as used herein, means —C(H)(═O).

The term “formylalkyl” as used herein, means a formyl group, as defined herein, appended to the parent moiety through an alkyl group as defined herein.

The term “halo” or “halogen” as used herein, means —Cl, —Br, —I or —F.

The term “haloalkoxy” as used herein, refers to an alkoxy group, as defined herein, in which one, two, three or four hydrogen atoms are replaced by halogen. Representative examples of haloalkoxy include, but are not limited to, chloromethoxy, 2-fluoroethoxy, trifluoromethoxy, 2-chloro-3-fluoropentyloxy, and pentafluoroethoxy.

The term “haloalkoxy” as used herein, refers to an haloalkoxy group, as defined herein, appended to the parent moiety through an alkyl group, as defined herein.

The term “haloalkyl” as used herein, refers to an alkyl group, as defined herein, in which one, two, three or four hydrogen atoms are replaced by halogen. Representative examples of haloalkyl include, but are not limited to, chloromethyl, 2-fluoroethyl, trifluoromethyl, pentafluoroethyl, and 2-chloro-3-fluoropentyl.

The term “heterocycle” or “heterocyclic” as used herein, refers to a monocyclic or bicyclic, non-aromatic, saturated or partially unsaturated ring system. Monocyclic ring systems are exemplified by a 4-membered ring containing one heteroatom independently selected from oxygen, nitrogen and sulfur; or a 5-, 6-, 7-, or 8-membered ring containing one, two or three heteroatoms wherein the heteroatoms are independently selected from nitrogen, oxygen and sulfur. The 5-membered ring has 0 or 1 double bond. The 6-memebered ring has 0, 1 or 2 double bonds. The 7- or 8-membered ring has 0, 1, 2 or 3 double bonds. Representative examples of monocyclic ring systems include, but are not limited to, azetidinyl, azepanyl, azepinyl, diazepinyl, dioxolanyl, dioxanyl, dithianyl, imidazolinyl, imidazolidinyl, isothiazolinyl, isothiazolidinyl, isoxazolinyl, isoxazolidinyl, morpholinyl, 3-oxo-morpholinyl, oxadiazolinyl, oxadiazolidinyl, oxazolinyl, 2-oxo-oxazolinyl, oxazolidinyl, piperazinyl, piperidyl, pyranyl, pyrazolinyl, pyrazolidinyl, pyrrolinyl, pyrrolidinyl, tetrahydrofuryl, tetrahydropyranyl, tetrahydropyridyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, 1,4-diazepanyl and trithianyl. Bicyclic ring systems are exemplified by any of the above monocyclic ring systems fused to a phenyl group, a monocyclic cycloalkenyl group, as defined herein, a monocyclic cycloalkyl group, as defined herein, or an additional monocyclic heterocycle group, as defined herein. Representative examples of bicyclic ring systems include but are not limited to, benzodioxinyl, benzodioxolyl, benzopyranyl, benzothiopyranyl, 2,3-dihydroindol-3-yl, 2,3-dihydrobenzofuran-3-yl, 2,3-dihydrobenzothien-3-yl, 2,3-dihydroisoindol-3-yl, 1,3-dihydro-isobenzofuran-3-yl, 1,3-dihydro-benzo[c]thien-3-yl, tetrahydroisoquinolinyl, tetrahydroquinolinyl, 3-azabicyclo[3.2.0]heptyl, 3,6-diazabicyclo[3.2.0]heptyl, octahydrocyclopenta[c]pyrrolyl, hexahydro-1H-furo[3,4-c]pyrrolyl, and octahydropyrrolo[3,4-c]pyrrolyl. The monocyclic or bicyclic ring systems as defined herein may have two of the non-adjacent carbon atoms connected by a heteroatom selected from nitrogen, oxygen or sulfur, or an alkylene bridge of between one and three additional carbon atoms. Representative examples of monocyclic or bicyclic ring systems that contain such connection between two non-adjacent carbon atoms include, but not limited to, 2-azabicyclo[2.2.2]octyl, 2-oxa-5-azabicyclo[2.2.2]octyl, 2,5-diazabicyclo[2.2.2]octyl, 2-azabicyclo[2.2.1]heptyl, 2-oxa-5-azabicyclo[2.2.1]heptyl, 2,5-diazabicyclo[2.2. I]heptyl, 2-azabicyclo[2.1.1]hexyl, 5-azabicyclo[2.1.1]hexyl, 3-azabicyclo[3.1.1]heptyl, 6-oxa-3-azabicyclo[3.1.1]heptyl, 8-azabicyclo[3.2.1]octyl, 8-azabicyclo[3.2.1]oct-8-yl, 3-oxa-8-azabicyclo[3.2.1]octyl, 1,4-diazabicyclo[3.2.2]nonyl, 1,4-diazatricyclo[4.3.1.1^{3,8}]undecyl, 3,10-diazabicyclo[4.3.1]decyl, or 8-oxa-3-azabicyclo[3.2.1]octyl, octahydro-1H-4,7-methanoisoindolyl, and octahydro-1H-4,7-epoxyisoindolyl. The heterocycle groups of the invention are substituted or unsubstituted, and are connected to the parent molecular moiety through any substitutable carbon or nitrogen atom in the groups. The nitrogen heteroatom may or may not be quaternized, and the nitrogen or sulfur heteroatom may or may not be oxidized. In addition, the nitrogen containing heterocyclic rings may or may not be N-protected.

The term “heteroaryl” as used herein, refers to an aromatic five- or six-membered ring where at least one atom is selected from the group consisting of N, O, and S, and the remaining atoms are carbon. The five membered rings have two double bonds, and the six membered rings have three double bonds. The term “heteroaryl” also includes bicyclic systems where a monocyclic heteroaryl ring is fused to a phenyl group, a monocyclic cycloalkyl group, as defined herein, a monocyclic cycloalkenyl group, as defined herein, a monocyclic heterocycle group, as defined herein, or an additional monocyclic heteroaryl group. Representative examples of heteroaryl groups include, but not limited to, benzothienyl, benzoxazolyl, benzimidazolyl, benzoxadiazolyl, 6,7-dihydro-1,3-benzothiazolyl, furyl, imidazolyl, imidazo[1,2-a]pyridinyl, indazolyl, indolyl, isoindolyl, isoxazolyl, isoquinolinyl, isothiazolyl, naphthyridinyl, oxadiazolyl, oxazolyl, pyridoimidazolyl, pyridyl, pyridazinyl, pyrimidinyl, pyrazinyl, pyrazolyl, pyrrolyl, quinolinyl, thiazolyl, thienyl, triazolyl, thiadiazolyl, tetrazolyl, 1,2,3,4-tetrahydro-1,8-naphthyridin-2-yl, and 5,6,7,8-tetrahydroquinolin-5-yl. The heteroaryl groups of the present invention can be substituted or unsubstituted, and are connected to the parent molecular moiety through any substitutable carbon or nitrogen atom in the groups. In addition, the nitrogen heteroatom may or may not be quaternized, the nitrogen and the sulfur atoms in the group may or may not be oxidized. Also, the nitrogen containing rings may or may not be N-protected.

The term “heteroarylalkyl” as used herein, means an heteroaryl group, as defined herein, appended to the parent molecular moiety through an alkyl group, as defined herein.

The term “heteroatom” as used herein, refers to nitrogen, oxygen or sulfur atom.

The term “hydroxy” or “hydroxyl” as used herein, means an —OH group.

The term “hydroxyalkyl” as used herein, refers to an alkyl group, as defined herein, in which one or two hydrogen atoms are replaced by a hydroxyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “nitro” as used herein, refers to an —NO₂ group.

The term “nitroalkyl” as used herein, refers to an nitro group, as defined herein, appended to the parent moiety through an alkyl group, as defined herein.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. The salts can be prepared in situ during the final isolation and purification of the compounds of the invention or separately by reacting a free base function with a suitable organic acid.

The term “pharmaceutically acceptable prodrug” or “prodrug,” as used herein, represents those prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of formula (I), for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

### EMBODIMENTS OF THE INVENTION

In its principal embodiment, the present invention relates to a method of treating neuropathic pain, chronic inflammatory pain, inflammation, neurodegeneration and of promoting neuroregeneration comprising administering a therapeutically effective amount of a compound that is a selective P2X₇ receptor antagonist of formula I 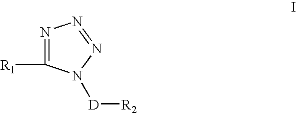
or a pharmaceutically acceptable salt, prodrug, or salt of a prodrug thereof, as described above. More particularly, the method comprises the use of compounds of formula (I), which include, but are not limited to, compounds wherein D is C₁₋₅ alkylene, R₁ and R₂ are aryl groups. Prefered compounds comprise those in which D is C₁₋₅ alkylene, R₁ is phenyl and R₂ is phenyl. The methods of the present invention also include compounds in which D is C₁₋₅ alkylene, R₁ is phenyl and R₂ is heteroaryl, in which R₂ can be selcted from the groups consisting of of pyridinyl, thiazolyl, imidazolyl, isoxazolyl, thienyl and quinolinyl. Preferably, the methods of the present invention comprise compounds in which D is C₁₋₅ alkylene, R₁ is phenyl and R₂ is pyridinyl. Preferred compounds also include those in which D is C₁₋₅ alkylene, R₁ is phenyl and R₂ is thiazolyl. Other preferred compounds contemplated for the method of the present invention include those in which D is C₁₋₅ alkylene, R₁ is phenyl and R₂ is imidazolyl. Preferred compounds also include those in which D is C₁₋₅ alkylene, R₁ is phenyl and R₂ is isoxazolyl, and those in which D is C₁₋₅ alkylene, R₁ is phenyl and R₂ is thienylyl. Other preferred compounds contemplated for the method of the present invention include those in which D is C₁₋₅ alkylene, R₁ is phenyl and R₂ is quinolinyl. The method of the present invention also includes compounds in which D is C₁₋₅ alkylene, R₁ is heteroaryl and R₂ is aryl, preferably, R₁ is selected form the group consisting of pyridinyl and thienyl, and and R₂ is phenyl. Preferred compounds contemplated for the method of the present invention also include those in which D is a bond, R₁ is aryl and R₂ is heteroaryl. Preferred compounds also include those in which D is a bond, R₁ is phenyl and R₂ is pyridinyl. Other preferred compounds include compounds in which D is a bond, R₁ is heteroarylalkyl and R₂ is aryl.

The compounds of the invention can be used in the form of pharmaceutically acceptable salts, prodrugs, esters, or amides derived from inorganic or organic acids. The method of the invention contemplates the use of pharmaceutically active compounds either chemically synthesized or formed by in vivo biotransformation to compounds of formula (I).

The methods of the present invention are based on the fact that the compounds described herein are antagonists of the P2X₇ receptor.

In the central nervous system, the P2X₇ receptor is predominately expressed by microglia, the resident macrophages of the brain. Upregulation of the P2X₇ receptor, most likely on activated microglia, has been reported in association with ischemic damage and necrosis induced by occlusion of middle cerebral artery in rat brain (Collo G. et al. Neuropharmacology, Vol. 36, pages 1277-1283, 1997). Recent studies indicate a role of the P2X₇ receptor in the generation of superoxide in microglia, and an increase of the number of P2X₇ receptors around β-amyloid plaques in a transgenic mouse model for Alzheimer's disease (Parvathenani et al., J. Biol. Chemistry, Vol. 278, pages 13300-13317, 2003) and in multiple sclerosis lesions from autopsy brain sections (Narcisse et al., Glia, Vol. 49, pages 245-258 (2005). Activation of the P2X₇ receptor on cells of the immune system (macrophages, mast cells and lymphocytes) leads to release of interleukin-1β (IL-1β), giant cell formation, degranulation, and L-selectin shedding. ATP has been shown to increase local release and process of IL-1β following lipopolysaccharide S (LPS) intraperitoneal injections in rats through a P2X₇ receptor mediated mechanism (Griffiths et al., J. Immunology Vol. 154, pages 2821-2828 (1995); Solle et al., J. Biol. Chemistry, Vol. 276, pages 125-132, (2001)). Oxidized ATP (oATP), a nonselective and irreversible P2X₇ antagonist, was recently reported to possess peripherally mediated antinociceptive properties in inflamed rats (Dell'Antonio et al. Neuroscience Lett., Vol. 327, pages 87-90, 2002). Activation of P2X₇ receptors localized on presynaptic terminals in the central and peripheral nervous systems (Deuchars et al J. Neuroscience, Vol. 21, p7143-7152, 2001) induces release of the excitatory amino acid neurotransmitter glutamate. A link between a P2X₇ purinoceptor gene and chronic, inflammatory and neuropathic pain has also been reported (Hatcher et al., The 6th International Conference on the Mechanisms and Treatment of Neuropathic Pain. San Fransisco, Calif.—Sep. 18-20, 2003). These findings indicate a role for the P2X₇ receptor in the process of neuronal synaptic transmission and therefore a potential role for P2X₇ antagonists as novel therapeutic tool to treat neuropathic pain.

Antagonists to the P2X₇ receptor significantly improved functional recovery and decreased cell death in spinal cord injury (SCI) animal models. Rats with SCI were administered P2X₇ receptor irreversible antagonists oATP and PPADS with a resulting decrease of histological injury and improved recovery of motor function after the lesions (Wang et al., Nature Medicine Vol. 10, pages B21-B27, 2004).

Therefore, compounds that are antagonists of the P2X₇ receptor are useful in methods for the prevention, or treatment, of conditions associated with inflammatory pain, inflammation, neuropathic pain, and neurodegeneration. More particularly, are useful to promote neuroregeneration and to treat neurodegeneration underlying several progressive CNS disorders, including, but not limited to, Alzheimer's disease, Parkinson's disease, amyotrophic lateral sclerosis, Huntington's disease, dementia with Lewy bodies, multiple sclerosis as well as diminished CNS function resulting from traumatic brain injury.

Preparation of the Compounds

The reactions exemplified in the schemes are performed in a solvent appropriate to the reagents and materials employed and suitable for the transformations being effected. The described transformations may require modifying the order of the synthetic steps or selecting one particular process scheme over another in order to obtain a desired compound of the invention, depending on the functionality present on the molecule.

Nitrogen protecting groups can be used for protecting amine groups present in the described compounds. Such methods, and some suitable nitrogen protecting groups, are described in Greene and Wuts (Protective Groups In Organic Synthesis, Wiley and Sons, 1999). For example, suitable nitrogen protecting groups include, but are not limited to, tert-butoxycarbonyl (Boc), benzyloxycarbonyl (Cbz), benzyl (Bn), acetyl, and trifluoracetyl. More particularly, the Boc protecting group may be removed by treatment with an acid such as trifluoroacetic acid or hydrochloric acid. The Cbz and Bn protecting groups may be removed by catalytic hydrogenation. The acetyl and trifluoracetyl protecting groups may be removed by a hydroxide ion.

The compounds and intermediates of the invention may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

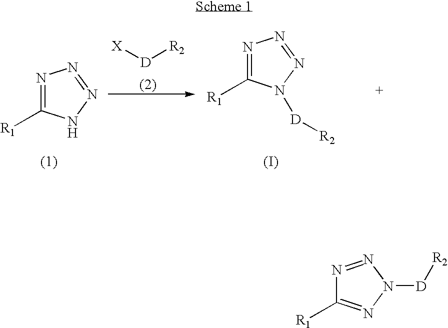

As shown in Scheme 1, tetrazole compounds of formula (I) wherein D is C₁₋₅ alkylene, and R₁, R₂ are as defined in formula (I) can be prepared from an NH-unsubstituted tetrazoles of formula (1) (either prepared by methodologies well known by those skilled in the art such as that illustrated in Scheme 5, or purchased) by reaction with an appropriate halides of formula (2) wherein X is Cl or Br, and a base like triethylamine or sodium hydride, in a solvent such as N,N-dimethylformamide, tetrahydrofuran or acetonitrile. The reaction is generally performed at a temperature from about room temperature to about 60° C., preferably at about room temperature, for a period of about 2 hours to about 24 hours. This reaction produces a mixture of regioisomers from with the desired 1,5 substituted regioisomer can be isolated by standard chromatographic methods. Alternatively, compounds of formula (I) can also be obtained from compounds of formula (1) by reaction with alcohols of formula (2) wherein X is OH, a phosphine such as triphenylphosphine, and diethylazodicarboxylate. The reaction is generally carried out in a solvent such as tetrahydrofuran, at a temperature from about 0° C. to about room temperature, preferably at room temperature, for a period of about 1 hour to about 24 hours.

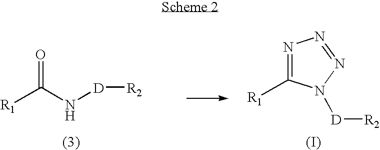

As shown in Scheme 2, tetrazole compounds of formula (I) can be prepared by reaction of a suitably substituted amide of formula (3) with (a) a chorinating agent like SOCl₂ or PCl₅ in a solvent such as, but not limited to, toluene, acetonitrile, tetrahydrofuran, and (b) by reaction the product of step (a) with an azide such as sodium azide or trimethylsilylazide in the presence of triethylamine and tetrabutylammonium bromide in a solvent such as acetonitrile. Catalytic amount of N,N-dimethylformamide is generally added to the reaction mixture if SOCl₂ is the chlorinating agent of choice in step (a), and the reaction is generally performed at a temperature from about room temperature to about 80° C., for a period of about 2 hours to about 10 hours. The reaction is feasible in the absence or presence of a base such as triethylamine. Step (b) is usually conducted at a temperature from about 10° C. to about room temperature, for a period of about 2 hours to about 24 hours.

Substituted amides of formula (3) can be purchased or prepared from acids of formula R₁COOH and amines of formula NH₂DR₂, wherein R₁, D and R₂ are as defined in formula (I), using standard transformations well known in the art. One example of such transformation is to (a) convert acids formula R₁COOH to chlorides of formula R₁COCl, by reaction with thionyl chloride, in the presence of N,N-dimethylformamide, in a solvent such as tetrahydrofuran, toluene, or acetonitrile, and (b) react product of step (a) with amines of formula NH₂DR₂ in the presence of a base such as, but not limited to, triethylamine. Step (a) is generally conducted in a solvent such as, but not limited to, toluene, N,N-dimethylformamide, tetrahydrofuran or acetonitrile, at a temperature from about room temperature to about 80° C., for a period of about 1 hour to about 6 hours. Step (b) can be carried out in a solvent such as, but not limited to, toluene, N,N-dimethylformamide, tetrahydrofuran or acetonitrile, at a temperature from about 0° C. to about room temperature, for a period of about 1 hour to about 24 hours.

Alternatively, the coupling of acids of formula R₁COOH with amines of formula NH₂DR₂ can also be made feasible in the presence of coupling reagents such as, but not limited to, 1-(3-dimethylaminopropyl)-3-ethylcarbodiimide hydrochloride (EDC) or 1,3-dicyclohexylcarbodiimide (DCC), optionally in the presence of 1-hydroxybenzotriazole hydrate (HOBT) or 3-hydroxy-1,2,3-benzotriazin-4(3H)-one (HOOBT), and optionally in the presence of a base such triethylamine. The reaction is generally performed at a temperature from about 0° C. to about 40° C., for a period of about 1 hour to about 24 hours, in a solvent such as, but not limited to, N,N-dimethylformamide, tetrahydrofuran, dichloromethane, and the like.

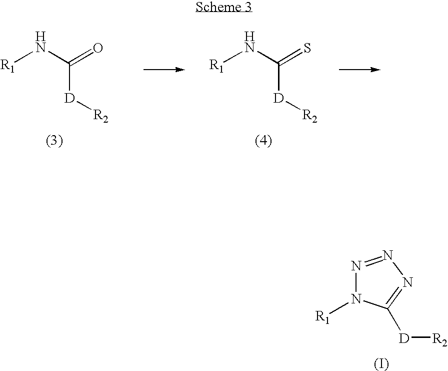

As shown in Scheme 3, amides of formula (3) can also be converted to tetrazole compounds of formula (I) via a substituted thioamide of formula (4). Thioamides of formula (4) can be obtained from reaction of amides of formula (3) (purchased or prepared by known methodologies) with Lawesson's reagent or an analog of Lawesson's reagent such as 2,4-bis(p-tolylthio)-1,3-dithia-2,4-diphosphethane-2,4-disulfide, in a solvent such as, but not limited to, toluene, tetrahydrofuran, acetonitrile or dichloromethane, at a temperature from about room temperature to about 80° C., for a period of about 2 hours to about 24 hours. Thioamides of formula (4) can be reacted with a mercury (II) salt such as, but not limited to, mercuric acetate and as mercury(II) chloride, and an azide like sodium azide or trimethylsilylazide in a solvent such as tetrahydrofuran at a temperature from about 0° C. to about room temperature for a period of about 1 hour to about 10 hours.

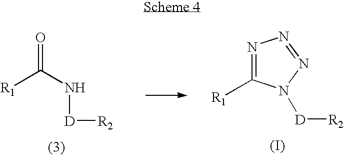

As shown in Scheme 4, tetrazole compounds of formula (I) wherein D is C₁₋₅ alkylene, R₁ is cycloalkyl, cycloalkenyl, heterocycle, aryl or heteroaryl, and R₂ is cycloalkyl, cycloalkenyl, heterocycle, or heteroaryl, can also be prepared from reaction of a suitably substituted amide of formula (3) (purchased or prepared by methods well known by those skilled in the art) with triphenylphosphine, an azodicarboxylate derivative such as, but not limited to, diisopropylazodicarboxylate, diethylazodicarboxylate, dimethylazodicarboxylate and dicyclohexylazodicarboxylate, and an azide such as, but not limited to, trimethylsilylazide and sodium azide in a solvent such as tetrahydrofuran. The reaction can be conducted at about room temperature for a period of about 24 hours.

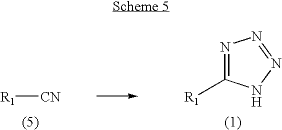

NH-unsubstituted tetrazoles of formula (1) wherein R₁ is as defined in formula (I) can be prepared from nitriles of formula (5) as shown in Scheme 5. Nitriles of formula (5) can be reacted with an azide such as, but not limited to, sodium azide or trimethylsilylazide, in a suitable solvent, at a temperature from about 50° C. to about 100° C., for a period of about 2 hours to about 24 hours. The reaction is generally carried out in the presence of a Lewis acid such as, but not limited to, trimethylaluminum, zinc bromide, or lithium chloride/ammonium chloride. Examples of the solvent employed include, but not limited to, N,N-dimethylformamide, toluene, water, isopropyl alcohol, or mixtures thereof. The reaction produces a mixture of regioisomers from which the desired isomer as depicted in formula (1) can be isolated by standard chromatography.

### EXAMPLES

The following Examples are intended as an illustration of and not a limitation upon the scope of the invention as defined in the appended claims.

### Example 1

### 1-(2,6-dichlorobenzyl)-5-(2,3-dichlorophenyl)-1H-tetraazole

### Example 1A

### 5-(2,3-dichlorophenyl)-1H-tetraazole

A 2.0 M solution of Al(CH₃)₃ in toluene (35 mL) was treated with 2,3-dichlorobenzonitrile (8.00 g, 37.2 mmol) followed by azidotrimethylsilane (5.14 g, 44.6 mmol) slowly and then the mixture was heated at 80° C. for 16 hours behind a blast shield. The mixture was cooled to 0° C. and treated with 2N HCl (100 mL) dropwise over 1 hour. The mixture was allowed to warm to ambient temperature and extracted twice with ethyl acetate (100 mL). The combined organic phases were dried over Na₂SO₄, filtered through a ½″ pad of silica gel, and the solvent was evaporated under reduced pressure. The residue was purified by recrystallization from ethyl acetate/hexanes to provide the title compound. MS (DCI/NH₃) m/z 215 (M)⁺; ¹H NMR (DMSO-d₆) δ 7.92 (dd, 1H, J=8.1, 1.7 Hz), 7.79 (dd, 1H, J=7.8, 1.7 Hz), 7.59 (t, 1H, J=8.0 Hz).

### Example 1B

### 1-(2,6-dichlorobenzyl)-5-(2,3-dichlorophenyl)-1H-tetraazole

5-(2,3-Dichlorophenyl)-1H-tetraazole (105 mg, 0.488 mmol) in dry DMF (3 mL) was treated with NaH (22 mg, 0.537 mmol) followed by addition of 2-bromomethyl-1,3-dichlorobenzene (187 mg, 0.708 mmol). The mixture was stirred for 16 hours, diluted with EtOAc, washed with 2N HCl, dried over Na₂SO₄, filtered, and the filtrate was evaporated under reduced pressure. The residue was purified by flash chromatography eluting with hexanes:EtOAc (3:1) to provide the title compound. MS (DCI/NH₃) m/z 375 (M+H)⁺; ¹H NMR (DMSO-d₆) δ 7.94 (dd, 1H, J=8.1, 1.7 Hz), 7.90 (dd, 1H, J=7.8, 1.7 Hz), 7.56 (t, 1H, J=8.0 Hz), 7.51-7.38 (m, 3H), 5.77 (s, 2H); Anal. calcd for C₁₄H₈ClN₄: C, 44.95; H, 2.16; N, 14.98. Found: C, 44.85; H, 2.14; N, 14.99.

### Example 2

### 1-benzyl-5-(2,3-dichlorophenyl)-1H-tetraazole

The title compound was prepared using the procedure described in Example 1B except using benzyl bromide instead of 2-bromomethyl-1,3-dichlorobenzene. MS (DCI/NH₃) m/z 305 (M)⁺; ¹H NMR (DMSO-d₆) δ 7.90 (dd, 1H, J=7.7, 2.2 Hz), 7.60-7.50 (m, 2H), 7.31-7.23 (m, 3H), 7.08-7.00 (m, 2H), 5.56 (s, 2H); Anal. calcd for C₁₄H₁₀Cl₂N₄: C, 55.10; H, 3.30; N, 18.36. Found: C, 54.91; H, 3.24; N, 18.43.

### Example 4

### 1-(4-chlorobenzyl)-5-(2,3-dichlorophenyl)-1H-tetraazole

The title compound was prepared using the procedure described in Example 1B except using 1-bromomethyl-4-chlorobenzene instead of 2-bromomethyl-1,3-dichlorobenzene. MS (DCI/NH₃) m/z 341 (M+H)⁺; ¹H NMR (DMSO-d₆) δ 7.93 (dd, 1H, J=7.8, 1.9 Hz), 7.61-7.54 (m, 2H), 7.35 (d, 2H, J=8.4 Hz), 7.10 (d, 2H, J=8.4 Hz), 5.58 (s, 2H); Anal. calcd for C₁₄H₉Cl₃N₄: C, 49.51; H, 2.67; N, 16.50. Found: C, 49.46; H, 2.58; N, 16.71.

### Example 5

### 1-(2,4-dichlorobenzyl)-5-(2,3-dichlorophenyl)-1H-tetraazole

The title compound was prepared using the procedure described in Example 1B except using 1-chloromethyl-2,4-dichlorobenzene instead of 2-bromomethyl-1,3-dichlorobenzene. MS (DCI/NH₃) m/z 375 (M+H)⁺; ¹H NMR (DMSO-d₆) δ 7.92 (ddd, 1H J=8.0, 1.5, 0.6 Hz), 7.65-7.54 (m, 3H), 7.35 (ddd, 1H, J=6.1, 2.2, 0.6 Hz), 7.24 (d, 1H, J=8.3 Hz), 5.67 (s, 2H); Anal. calcd for C₁₄H₈ClN₄: C, 44.95; H, 2.16; N, 14.98. Found: C, 44.88; H, 2.23; N, 14.94.

### Example 6

### 2-{[5-(2,3-dichlorophenyl)-1H-tetraazol-1-yl]methyl}pyridine

The title compound was prepared using the procedure described in Example 9 except using 2-bromomethyl pyridine hydrobromide instead of 4-bromomethylpyridine hydrobromide. MS (DCI/NH₃) m/z 306 (M-HCl)⁺; ¹H NMR (DMSO-d₆) δ 8.45 (ddd, 1H, J=4.9, 1.5, 0.9 Hz), 7.89 (dd, 1H J=8.3, 1.5 Hz), 7.81 (td, 1H, J=7.7, 1.8 Hz), 7.62 (dd, 1H, J=7.7, 1.5 Hz), 7.52 (t, 1H, J=7.8 Hz), 7.38-7.30 (m, 2H), 5.76 (s, 2H); Anal. calcd for C₁₃H₉Cl₂N₅.HCl: C, 45.57; H, 2.94; N, 20.44. Found: C, 45.53; H, 2.81; N, 20.61.

### Example 7

### 3-{[5-(2,3-dichlorophenyl)-1H-tetraazol-1-yl]methyl}pyridine

5-(2,3-Dichlorophenyl)-1H-tetraazole (2.0 g, 9.3 mmol) and triethylamine (2.35 g, 23.2 mmol) in MeCN (20 mL) were treated with 3-bromomethylpyridine hydrobromide (2.35 g, 9.3 mmol) and stirred at ambient temperature for 24 hours. The mixture was diluted with EtOAc, washed with brine, and absorbed onto silica gel. The product was purified by flash chromatography on silica gel eluting with EtOAc:hexanes:EtOH (1:1:0.1) to provide the title compound. The title compound was converted to the hydrochloride salt with ethanolic HCl. MS (DCI/NH₃) m/z 306 (M-HCl)⁺; ¹H NMR (DMSO-d₆) δ 8.70 (dd, 1H, J=5.1, 1.4 Hz), 8.57 (d, 1H, J=2.0 Hz), 7.99-7.92 (m, 2H), 7.72-7.56 (m, 3H), 5.74 (s, 2H); Anal. calcd for C₁₃H₉Cl₂N₅.HCl: C, 45.57; H, 2.94; N, 20.44. Found: C, 45.44; H, 2.92; N, 20.26.

### Example 8

### 1-(2-chlorobenzyl)-5-(2,3-dichlorophenyl)-1H-tetraazole

The title compound was prepared using the procedure described in Example 1B except using 1-bromomethyl-2-chlorobenzene instead of 2-bromomethyl-1,3-dichlorobenzene. MS (DCI/NH₃) m/z 341 (M+H)⁺; ¹H NMR (DMSO-d₆) δ 7.90 (dd, 1H, J=8.0, 1.5 Hz), 7.62 (dd, 1H, J=7.7, 1.5 Hz), 7.55 (t, 1H, J=7.8 Hz), 7.41 (dd, 1H, J=8.0, 1.5 Hz), 7.35 (td, 1H, J=7.1, 1.8 Hz), 7.25 (td, 1H, J=7.4, 1.5 Hz), 7.20 (dd, 1H, J=8.0, 1.8 Hz), 5.68 (s, 2H); Anal. calcd for C₁₄H₉Cl₃N₄: C, 49.51; H, 2.67; N, 16.50. Found C, 49.63; H, 2.68; N, 16.47.

### Example 9

### 4-{[5-(2,3-dichlorophenyl)-1H-tetraazol-1-yl]methyl}pyridine

5-(2,3-Dichlorophenyl)-1H-tetraazole (133 mg, 0.618 mmol), 4-bromomethylpyridine hydrobromide (203 mg, 0.804 mmol), and triethylamine (0.165 mL, 1.91 mmol) were dissolved in MeCN (7 mL) and stirred for 16 hours. The solvent was evaporated under reduced pressure and the mixture was purified by reverse phase HPLC (gradient elution with 10-100% MeCN containing 0.01M aq. NH₄OAc) to provide the title compound. The title compound was converted to the hydrochloride salt with ethanolic HCl. MS (DCI/NH₃) m/z 306 (M-HCl)⁺; ¹H NMR (DMSO-d₆) δ 8.66 (d, 2H, J=6.1 Hz), 7.94 (dd, 1H, J=8.1, 1.7 Hz), 7.66 (dd, 1H, J=7.8, 1.7 Hz), 7.57 (t, 1H, J=8.0 Hz), 7.37 9d, 2H, J=6.1 Hz), 5.80 (s, 2H); Anal. calcd for C₁₃H₉Cl₂N₅.HCl.0.75H₂O: C, 43.84; H, 3.25; N, 19.67. Found: C, 44.02; H, 3.20; N, 19.45.

### Example 10

### 5-(2,3-dichlorophenyl)-1-(2-methoxybenzyl)-1H-tetraazole

5-(2,3-Dichlorophenyl)-1H-tetraazole (133 mg, 0.618 mmol), 1-chloromethyl-2-methoxybenzene (117 mg, 0.743 mmol), and triethylamine (0.137 mL, 0.989 mmol) were dissolved in MeCN (7 mL) and stirred for 16 hours. The solvent was evaporated under reduced pressure and the mixture was purified by reverse phase HPLC (gradient elution with 10-100% MeCN containing 0.1M aqueous TFA) to provide the title compound. MS (DCI/NH₃) m/z 335 (M)⁺; ¹H NMR (DMSO-d₆) δ 7.92 (dd, 1H J=7.1, 3.1 Hz), 7.63-7.53 (m, 2H), 7.34-7.24 (m, 1H), 7.13 (dd, 1H, J=7.5, 1.0 Hz), 6.94-6.81 (m, 2H), 5.47 (s, 2H), 3.53 (s, 3H); Anal. calcd for C₁₅H₁₂Cl₂N₄O.0.3H₂O: C, 52.80; H, 3.74; N, 16.42. Found: C, 52.86; H, 3.57; N, 16.42.

### Example 11

### 5-(2,3-dichlorophenyl)-1-(2-methoxybenzyl)-1H-tetraazole

The title compound was prepared using the procedure described in Example 10 except using 1-chloromethyl-4-methoxybenzene instead of 1-chloromethyl-2-methoxybenzene. MS (DCI/NH₃) m/z 335 (M)⁺; ¹H NMR (DMSO-d₆) δ 7.93 (dd, 1H J=5.1, 4.4 Hz), 7.58 (s, 1H), 7.56 (d, 1H, J=1.0 Hz), 6.99 (d, 2H, J=8.8 Hz), 6.82 (d, 2H, J=8.8 Hz), 5.49 (s, 2H); 3.71 (s, 3H); Anal. calcd for C₁₅H₁₂Cl₂N₄O: C, 53.75; H, 3.61; N, 16.72. Found: C, 53.76; H, 3.54; N, 16.84.

### Example 12

### methyl 4-{[5-(2,3-dichlorophenyl)-1H-tetraazol-1-yl]methyl}benzoate

The title compound was prepared using the procedure described in Example 10 except using methyl 4-(bromomethyl)benzoate instead of 1-chloromethyl-2-methoxybenzene. MS (DCI/NH₃) m/z 363 (M)⁺; ¹H NMR (DMSO-d₆) δ 7.92 (dd, 1H J=7.5, 2.0 Hz), 7.87 (d, 2H, J=8.5 Hz), 7.60 (dd, 1H, J=7.8, 2.0 Hz), 7.55 (t, 1H, J=7.6 Hz), 7.21 (d, 2H, J=8.1 Hz), 5.69 (s, 2H), 3.84 (s, 3H); Anal. calcd for C₁₆H₁₂Cl₂N₄O₂: C, 52.91; H, 3.33; N, 15.43. Found: C, 52.76; H, 3.20; N, 15.55.

### Example 13

### 5-(2,3-dichlorophenyl)-1-(2-fluorobenzyl)-1H-tetraazole

The title compound was prepared using the procedure described in Example 10 except using 1-bromomethyl-2-fluorobenzene instead of 1-chloromethyl-2-methoxybenzene. MS (DCI/NH₃) m/z 323 (M)⁺; ¹H NMR (DMSO-d₆) δ 7.92 (dd, 1H J=7.5, 2.0 Hz), 7.64 (dd, 1H, J=7.8, 1.7 Hz), 7.57 (t, 1H, J=7.8 Hz), 7.43-7.33 (m, 1H), 7.20-7.07 (m, 3H), 5.63 (s, 2H); Anal. calcd for C₁₄H₉Cl₂FN₄: C, 52.03; H, 2.81; N, 17.34. Found: C, 51.86; H, 2.72; N, 17.35.

### Example 14

### 5-(2,3-dichlorophenyl)-1-(2-bromobenzyl)-1H-tetraazole

The title compound was prepared using the procedure described in Example 10 except using 1-bromomethyl-2-bromobenzene instead of 1-chloromethyl-2-methoxybenzene. MS (DCI/NH₃) m/z 384 (M+H)⁺; ¹H NMR (DMSO-d₆) δ 7.90 (dd, 1H J=7.8, 1.7 Hz), 7.63 (dd, 1H, J=7.5, 1.7 Hz), 7.60-7.51 (m, 2H), 7.34-7.23 (m, 2H), 7.19 (dd, 1H, J=6.8, 2.7 Hz), 5.66 (s, 2H); Anal. calcd for C₁₄H₉Cl₂BrN₄: C, 43.78; H, 2.36; N, 14.59. Found: C, 43.81; H, 2.19; N, 14.62.

### Example 15

### 5-(2,3-dichlorophenyl)-1-(2-methylbenzyl)-1H-tetraazole

The title compound was prepared using the procedure described in Example 10 except using 1-bromomethyl-2-methylbenzene instead of 1-chloromethyl-2-methoxybenzene. MS (DCI/NH₃) m/z 319 (M)⁺; ¹H NMR (DMSO-d₆) δ 7.89 (dd, 1H J=7.8, 1.7 Hz), 7.61 (dd, 1H, J=7.8, 1.7 Hz), 7.53 (t, 1H, J=7.8 Hz), 7.22-7.11 (m, 2H), 7.01 (td, 1H J=7.5, 1.7 Hz), 6.75 (d, 1H, J=7.5 Hz), 5.6 (s, 2H), 2.08 (s, 3H); Anal. calcd for C₁₅H₁₂Cl₂N₄: C, 56.44; H, 3.79; N, 17.55. Found: C, 56.16; H, 3.75; N, 17.60.

### Example 16

### 1-(1,1′-biphenyl-2-ylmethyl)-5-(2,3-dichlorophenyl)-1H-tetraazole

The title compound was prepared using the procedure described in Example 10 except using 2-(bromomethyl)-1,1′-biphenyl instead of 1-chloromethyl-2-methoxybenzene. MS (DCI/NH₃) m/z 381 (M)⁺; ¹H NMR (DMSO-d₆) δ 7.85 (dd, 1H J=8.1, 1.4 Hz), 7.43-7.28 (m, 6H), 7.22 (dd, 1H, J=7.8, 1.4 Hz), 7.17 (dd, 1H, J=5.1, 1.7 Hz), 7.14 (dd, 1H, J=5.1, 1.6 Hz), 7.08-7.01 (m, 2H), 5.56 (s,2H); Anal. calcd for C₂₀H₁₄Cl₂N₄: C, 63.01; H, 3.70; N, 14.70. Found: C, 62.96; H, 3.76; N, 14.45.

### Example 17

### 5-(2,3-dichlorophenyl)-1-(2-iodobenzyl)-1H-tetraazole

The title compound was prepared using the procedure described in Example 10 except using 1-bromomethyl-2-iodobenzene instead of 1-chloromethyl-2-methoxybenzene. MS (DCI/NH₃) m/z 431 (M)⁺; ¹H NMR (DMSO-d₆) δ 7.90 (dd, 1H J=8.1, 1.7 Hz), 7.80 (dd, 1H, J=7.8, 1.4 Hz), 7.60 (dd, 1H, J=7.5, 1.7 Hz), 7.53 (t, 1H, J=7.8 Hz), 7.30 (td, 1H, J=7.5, 1.4 Hz), 7.11 (dd, 1H, J=7.8, 1.4 Hz), 7.05 (td, 1H, J=7.8, 1.7 Hz), 5.63 (s, 2H); Anal. calcd for C₁₄H₉Cl₂IN₄: C, 39.01; H, 2.10; N, 13.00. Found: C, 39.01; H, 2.15; N, 13.02.

### Example 18

### 1-benzyl-5-(2-chlorophenyl)-1H-tetraazole

### Example 18A

### 5-(2-chlorophenyl)-1H-tetraazole

The title compound was prepared using the procedure described in Example 1A except using 2-chlorobenzonitrile instead of 2,3-dichlorobenzonitrile.

### Example 18B

### 1-benzyl-5-(2-chlorophenyl)-1H-tetraazole

The title compound was prepared using the procedure described in Example 10 except using 5-(2-chlorophenyl)-1H-tetraazole and benzyl bromide instead of 5-(2,3-dichlorophenyl)-1H-tetraazole and 1-chloromethyl-2-methoxybenzene. MS (DCI/NH₃) m/z 271 (M)⁺; ¹H NMR (DMSO-d₆) δ 7.72-7.63 (m, 2H), 7.62-7.50 (m, 2H), 7.31-7.25 (m, 3H), 7.07-7.00 (m, 2H), 5.55 (s, 2H); Anal. calcd for C₁₄H₁₁ClN₄: C, 62.11; H, 4.10; N, 20.70. Found: C, 62.18; H, 4.01; N, 20.74.

### Example 19

### 1-(2-chlorobenzyl)-5-(2-chlorophenyl)-1H-tetraazole

The title compound was prepared using the procedure described in Example 10 except using 5-(2-chlorophenyl)-1H-tetraazole and 1-bromomethyl-2-chlorobenzene instead of 5-(2,3-dichlorophenyl)-1H-tetraazole and 1-chloromethyl-2-methoxybenzene. MS (DCI/NH₃) m/z 305 (M)⁺; ¹H NMR (DMSO-d₆) δ 7.68-7.60 (m, 3H), 7.56-7.50 (m, 1H), 7.41 (dd, 1H, J=7.8, 1.4 Hz), 7.35 (td, 1H, J=6.8, 1.7 Hz), 7.27 (td, 1H, J=7.5, 1.4 Hz), 7.22 (dd, 1H, J=7.5, 1.7 Hz), 5.65 (s, 2H); Anal. calcd for C₁₄H₁₀Cl₂N₄: C, 55.10; H, 3.30; N, 18.36. Found: C, 55.22; H, 3.18; N, 18.47.

### Example 20

### 5-(2-chlorophenyl)-1-(2,6-dichlorobenzyl)-1H-tetraazole

The title compound was prepared using the procedure described in Example 10 except using 5-(2-chlorophenyl)-1H-tetraazole and 2-bromomethyl-1,3-dichlorobenzene and instead of 5-(2,3-dichlorophenyl)-1H-tetraazole and 1-chloromethyl-2-methoxybenzene. MS (DCI/NH₃) m/z 341 (M+H)⁺; ¹H NMR (DMSO-d₆) δ 7.75-7.65 (m, 3H), 7.60-7.53 (m, 1H), 7.51-7.47 (m, 2H), 7.46-7.39 (m, 1H), 5.72 (s, 2H); Anal. calcd for C₁₄H₉Cl₃N₄: C, 49.51; H, 2.67; N, 16.50. Found: C, 49.62; H, 2.55; N, 16.59.

### Example 21

### 1-(4-chlorobenzyl)-5-(2-chlorophenyl)-1H-tetraazole

The title compound was prepared using the procedure described in Example 10 except using 5-(2-chlorophenyl)-1H-tetraazole and 1-bromomethyl-4-chlorobenzene instead of 5-(2,3-dichlorophenyl)-1H-tetraazole and 1-chloromethyl-2-methoxybenzene. MS (DCI/NH₃) m/z 305 (M)⁺; ¹H NMR (DMSO-d₆) δ 7.75-7.65 (m, 4H), 7.35 (d, 2H, J=8.5 Hz), 7.08 (d, 2H, J=8.5 Hz), 5.56 (s, 2H); Anal. calcd for C₁₄H₁₀Cl₂N₄: C, 55.10; H, 3.30; N, 18.36. Found: C, 54.93; H, 3.21; N, 18.38.

### Example 22

### 5-(2-chlorophenyl)-1-(2,4-dichlorobenzyl)-1H-tetraazole

The title compound was prepared using the procedure described in Example 10 except using 5-(2-chlorophenyl)-1H-tetraazole and 1-bromomethyl-2,4-dichlorobenzene instead of 5-(2,3-dichlorophenyl)-1H-tetraazole and 1-chloromethyl-2-methoxybenzene. MS (DCI/NH₃) m/z 341 (M+H)⁺; ¹H NMR (DMSO-d₆) δ 7.69-7.60 (m, 4H), 7.58-7.51 (m, 1H), 7.38 (dd, 1H, J=8.5, 2.0 Hz), 7.27 (d, 1H, J=8.5 Hz), 5.64 (s, 2H); Anal. calcd for C₁₄H₉Cl₃N₄: C, 49.51; H, 2.67; N, 16.50. Found: C, 49.26; H, 2.59; N, 16.43.

### Example 23

### 3-{[5-(2-chlorophenyl)-1H-tetraazol-1-yl]methyl}pyridine

The title compound was prepared using the procedure described in Example 9 except using 5-(2-chlorophenyl)-1H-tetraazole and 3-bromomethylpyridine hydrobromide instead of 5-(2,3-dichlorophenyl)-1H-tetraazole and 4-bromomethylpyridine hydrobromide. MS (DCI/NH₃) m/z 272 (M-HCl)⁺; ¹H NMR (DMSO-d₆) δ 8.67 (dd, 1H, J=5.1, 1.4 Hz), 8.53 (d, 1H, J=2.0 Hz), 7.90 (dt, 1H, J=8.1, 1.7 Hz), 7.72-7.54 (m, 5H), 6.88 (br s, 1H, HCl), 5.71 (s, 2H).

### Example 24

### 4-{[5-(2-chlorophenyl)-1H-tetraazol-1-yl]methyl}pyridine

The title compound was prepared using the procedure described in Example 9 except using 5-(2-chlorophenyl)-1H-tetraazole instead of 5-(2,3-dichlorophenyl)-1H-tetraazole. MS (DCI/NH₃) m/z 272 (M-HCl)⁺; ¹H NMR (DMSO-d₆) δ 8.67 (d, 2H, J=5.4 Hz), 7.71-7.63 (m, 3H), 7.58-7.51 (m, 1H), 7.41 (d, 2H, J=6.11 Hz), 5.80 (s, 2H), 5.41 (br s, 1H, HCl).

### Example 25

### 3-[1-(2-chlorobenzyl)-1H-tetraazol-5-yl]pyridine

### Example 25A

### 3-(1H-tetraazol-5-yl)pyridine

3-Cyanopyridine (1.0 g, 9.61 mmol), sodium azide (0.843 g, 12 mmol), ammonium chloride (0.694 g, 12 mmol), and lithium chloride (10 mg) were combined in DMF (10 mL) and heated at reflux behind a blast shield for 24 hours. The mixture was allowed to cool to ambient temperature, diluted with EtOAc, and washed with water. The title compound was recrystallized from EtOAc/hexanes. MS (DCI/NH₃) m/z 148 (M+H)⁺; ¹H NMR (DMSO-d₆) δ 9.22 (d, 1H, J=1.7 Hz), 8.77 (dd, 1H, J=4.7, 1.7 Hz), 8.40 (dt, 1H J=8.5, 2.0 Hz), 7.65 (ddd, 1H, J=7.8, 4.8, 1.0 Hz).

### Example 25B

### 3-[1-(2-chlorobenzyl)-1H-tetraazol-5-yl]pyridine

The title compound was prepared using the procedure described in Example 9 except using 3-(1H-tetraazol-5-yl)pyridine and 1-bromomethyl-2-chlorobenzene instead of 5-(2,3-dichlorophenyl)-1H-tetraazole and 4-bromomethylpyridine hydrobromide. MS (DCI/NH₃) m/z 272 (M-HCl)⁺; ¹H NMR (DMSO-d₆) δ 8.94 (d, 1H, J=1.4 Hz), 8.81 (dd, 1H, J=4.8, 1.7 Hz), 8.22 (ddd, 1H J=8.1, 2.4, 1.7 Hz), 7.65 (ddd, 1H, J=7.8, 4.8, 1.0 Hz), 7.47 (dd, 1H, J=7.8, 1.7 Hz), 7.38 (dt, 1H, J=7.5, 2.0 Hz), 7.33 (dt, 1H, J=7.1, 1.4 Hz), 7.25 (dd, 1H, J=7.5, 2.0 Hz), 5.86 (s, 2H).

### Example 26

### 1-(2-chlorobenzyl)-5-(2,3-dimethoxyphenyl)-1H-tetraazole

### Example 26A

### 5-(2,3-dimethoxyphenyl)-1H-tetraazole

2,3-Dimethoxybenzonitrile (2.0 g, 12.3 mmol), ZnBr₂ (2.8 g, 12.3 mmol), and sodium azide (0.88 g, 13.5 mmol) were suspended in H₂O (25 mL) and iPrOH (3 mL) and heated behind a blast shield at reflux for 24 hours. The mixture was allowed to cool to ambient temperature and diluted with EtOAc and 2N HCl. The aqueous phase was back extracted with EtOAc and the combined organic phases were dried over Na₂SO₄, filtered through a ¼″ silica gel plug, and the filtrate was concentrated under reduced pressure. The residue was purified by trituration with diethyl ether to provide the title compound. MS (DCI/NH₃) m/z 207 (M+H)⁺, m/z 181 (M+NH₄—HN₃)⁺.

### Example 26B

### 1-(2-chlorobenzyl)-5-(2,3-dimethoxyphenyl)-1H-tetraazole

The title compound was prepared using the procedure described in Example 10 except using 5-(2,3-dimethoxyphenyl)-1H-tetraazole and 1-bromomethyl-2-chlorobenzene instead of 5-(2,3-dichlorophenyl)-1H-tetraazole and 1-chloromethyl-2-methoxybenzene. MS (DCI/NH₃) m/z 331 (M)⁺; ¹H NMR (DMSO-d₆) δ 7.46-7.16 (m, 6H), 7.02 (dd, 1H, J=7.5, 1.4 Hz), 5.59 (s, 2H), 3.88 (s, 3H), 3.60 (s, 3H); Anal. calcd for C₁₆H₁₅ClN₄O₂: C, 58.10; H, 4.57; N, 16.94. Found: C, 58.23; H, 4.61; N, 17.08.

### Example 27

### 1-(2-chlorobenzyl)-5-(2,3-dimethoxyphenyl)-1H-tetraazole

The title compound was prepared using the procedure described in Example 10 except using 5-(2,3-dimethoxyphenyl)-1H-tetraazole instead of 5-(2,3-dichlorophenyl)-1H-tetraazole. MS (DCI/NH₃) m/z 327 (M+H)⁺; ¹H NMR (DMSO-d₆) δ 7.37-7.22 (m, 3H), 7.08 (dd, 1H, J=7.5, 1.4 Hz), 7.00 (dd, 1H, J=7.8, 1.7 Hz), 6.95-6.84 (m, 2H), 5.42 (s, 2H), 3.89 (s, 3H), 3.61 (s, 3H), 3.53 (s, 3H); Anal. calcd for C₁₇H₁₈N₄O₃: C, 62.57; H, 5.56; N, 17.17. Found: C, 62.51; H, 5.46; N, 17.25.

### Example 28

### 3-{[5-(2,3-dichlorophenyl)-1H-tetraazol-1-yl]methyl}-2-methylpyridine hydrochloride

### Example 28A

### 3-{[5-(2,3-dichlorophenyl)-1H-tetraazol-1-yl]methyl}-2-methylpyridine

5-(2,3-Dichlorophenyl)-1H-tetraazole (3.2 g, 14.88 mmol) and 3-(chloromethyl)-2-methylpyridine hydrochloride (2.65 g, 14.88 mmol) were combined in CH₃CN (25 mL), treated with triethylamine (6.23 mL, 44.64 mmol), and stirred at room temperature for 18 hours. The mixture was treated with brine (20 mL) and extracted with dichloromethane (2×40 mL). The organic layer was washed with saturated aqueous NaHCO₃ solution (15 mL), brine (15 mL), dried (Na₂SO₄), filtered, and the filtrate was concentrated under reduced pressure. The residue was purified by flash chromatography (elution with 1% ethanol, 49% ethyl acetate, 50% hexane) to provide the title compound. mp 121-122° C.; MS (ESI⁺) m/z 321 (M+H)⁺; ¹H NMR (DMSO-d₆) δ 8.44 (dd, 1H, J=1.7, 4.8 Hz), 7.83 (dd, 1H, J=1.7, 6.7 Hz), 7.81 (dd, 1H, J=1.7, 6.8 Hz), 7.65 (dd, 1H, J=1.7, 7.1 Hz), 7.52 (t, 1H, J=7.8 Hz), 7.27 (dd, 1H, J=4.7, 7.7 Hz), 6.10 (s, 2H), 2.52 (s, 3H); Anal. calcd for C₁₄H₁₁Cl₂N₅0.05CH₂Cl₂: C, 52.02; H, 3.45; N, 21.59. Found: C, 52.02; H, 3.43; N, 21.46.

### Example 28B

### 3-{[5-(2,3-dichlorophenyl)-1H-tetraazol-1-yl]methyl}-2-methylpyridine hydrochloride

3-{[5-(2,3-Dichlorophenyl)-1H-tetraazol-1-yl]methyl}-2-methylpyridine (1.13 g, 3.529 mmol) in dichloromethane:methanol (10 mL, 9:1) at ambient temperature was treated with 2.0 M hydrogen chloride in diethyl ether (2.12 mL, 4.235 mmol). The solvent was removed under reduced pressure leaving the hydrochloride salt, which was triturated with diethyl ether to provide the title compound. mp 104-105° C.; MS (ESI⁺) m/z 321 (M+H)⁺; ¹H NMR (DMSO-d₆) δ 8.60 (dd, 1H, J=1.7, 5.3 Hz), 7.95 (dd, 1H, J=1.7, 7.8 Hz), 7.70-7.67 (m, 1H), 7.70 (dd, 1H, J=1.7, 7.8 Hz), 7.59 (t, 1H, J=8.0 Hz), 7.49 (dd, 1H, J=5.42, 7.6 Hz), 5.77 (s, 2H), 2.47 (s, 3H); Anal. calcd for C₁₄H₁₂Cl₃N₅ 1.235H₂O: C, 44.38; H, 3.85; N, 18.48. Found: C, 44.78; H, 3.96; N, 18.08.

### Example 29

### 3-{[5-(2,3-dichlorophenyl)-1H-tetraazol-1-yl]methyl}-4-methylpyridine

The title compound was prepared using the procedure described in Example 28A except using 5-(chloromethyl)-4-methylpyridine hydrochloride instead of 3-(chloromethyl)-2-methylpyridine hydrochloride. mp 154-156° C.; MS (ESI⁺) m/z 321 (M+H)⁺; ¹H NMR (DMSO-d₆) δ 8.33 (d, 1H, J=3.0 Hz), 8.00 (s, 1H), 7.92 (dd, 1H, J=1.5, 8.0 Hz), 7.67 (dd, 1H, J=1.5, 7.6 Hz), 7.57 (t, 1H, J=7.8 Hz), 7.21 (d, 1H, J=5.1 Hz), 5.68 (s, 2H), 2.11 (s, 3H); Anal. calcd for C₁₄H₁₁Cl₂N₅: C, 52.52; H, 3.46; N, 21.87. Found: C, 52.78; H, 3.50; N, 21.92.

### Example 30

### 3-{[5-(2,3-dichlorophenyl)-1H-tetraazol-1-yl]methyl}-2,4-dimethylpyridine

The title compound was prepared using the procedure described in Example 28A except using 3-(chloromethyl)-2,4-dimethylpyridine hydrochloride instead of 3-(chloromethyl)-2-methylpyridine hydrochloride. mp 146-148° C.; MS (ESI⁺) m/z 335 (M+H)⁺; ¹H NMR (DMSO-d₆) δ 8.25 (d, 1H, J=5.1 Hz), 7.94 (dd, 1H, J=1.4, 8.1 Hz), 7.72 (dd, 1H, J=1.7, 7.6 Hz), 7.58 (t, 1H, J=7.8 Hz), 7.05 (d, 1H, J=4.8), 5.62 (s, 2H), 2.27 (s, 3H), 2.11 (s, 3H); Anal. calcd for C₁₅H₁₃Cl₂N₅: C, 53.91; H, 3.92; N, 20.96. Found: C, 53.65; H, 3.86; N, 20.71.

### Example 31

### 5-(2,3-dichlorophenyl)-1-(1,3-thiazol-4-ylmethyl)-1H-tetraazole

The title compound was prepared using the procedure described in Example 28A except using 4-(chloromethyl)-1,3-thiazole hydrochloride instead of 3-(chloromethyl)-2-methylpyridine hydrochloride. mp 153-154° C.; MS (ESI⁺) m/z 313 (M+H)⁺; ¹H NMR (DMSO-d₆) δ 9.00 (d, 1H, J=2.0 Hz), 7.92 (dd, 1H, J=1.7, 7.8 Hz), 7.62 (dd, 1H, J=1.7, 7.8 Hz), 7.58 (d, 1H, J=1.7), 7.58 (t, 1H, J=8.0 Hz), 5.73 (s, 2H); Anal. calcd for C₁₁H₇Cl₂N₅S: C, 42.32; H, 2.26; N, 22.43. Found: C, 42.20; H, 2.24; N, 22.14.

### Example 32

### 5-(2,3-dichlorophenyl)-1-[(2-methyl-1,3-thiazol-4-yl)methyl]-1H-tetraazole

The title compound was prepared using the procedure described in Example 28A except using 4-(chloromethyl)-2-methyl-1,3-thiazole hydrochloride instead of 3-(chloromethyl)-2-methylpyridine hydrochloride. mp 156-158° C.; MS (ESI⁺) m/z 327 (M+H)⁺; ¹H NMR (DMSO-d₆) δ 7.91 (dd, 1H, J=1.7, 7.8 Hz), 7.62 (dd, 1H, J=1.7, 7.8 Hz), 7.54 (t, 1H, J=7.8 Hz), 7.32 (s, 1H), 5.73 (s, 2H), 2.54 (s, 3H); Anal. calcd for C₁₂H₉Cl₂N₅S: C, 44.18; H, 2.78: N, 21.47. Found: C, 44.01; H, 2.58; N, 21.40.

### Example 33

### 5-(2,3-dichlorophenyl)-1-(1H-imidazol-4-ylmethyl)-1H-tetraazole

The title compound was prepared using the procedure described in Example 28A except using 4-(chloromethyl)-1H-imidazole hydrochloride instead of 3-(chloromethyl)-2-methylpyridine hydrochloride. MS (ESI⁺) m/z 296 (M+H)⁺; ¹H NMR (DMSO-d₆) δ 11.75 (brs, 1H), 7.94 (dd, 1H, J=1.7, 8.0 Hz), 7.81 (brs, 1H), 7.68 (dd, 1H, J=1.7, 7.8 Hz), 7.57 (t, 1H, J=8.0 Hz), 7.05 (s, 1H), 5.49 (s, 2H).

### Example 34

### ethyl 4-{[5-(2,3-dichlorophenyl)-1H-tetraazol-1-yl]methyl}isoxazole-3-carboxylate

The title compound was prepared using the procedure described in Example 28A except using ethyl 4-(chloromethyl)isoxazole-3-carboxylate instead of 3-(chloromethyl)-2-methylpyridine hydrochloride. mp 161-163° C.; MS (ESI⁺) m/z 369 (M+H)⁺; ¹H NMR (DMSO-d₆) δ 7.98 (dd, 1H, J=1.7, 8.1 Hz), 7.70 (dd, 1H, J=1.7, 7.8 Hz), 7.61 (t, 1H, J=7.8 Hz), 6.85 (s, 1H), 5.97 (s, 2H), 7.61 (q, 2H, J=7.1 Hz), 2.54 (t, 3H, J=7.1 Hz); Anal. calcd for C₁₄H₁₁Cl₂N₅O₃: C, 45.67; H, 3.01; N, 19.02. Found: C, 45.37; H, 2.90; N, 18.74.

### Example 35

### 5-(2,3-dichlorophenyl)-1-[(5-methyl-1H-imidazol-4-yl)methyl]-1H-tetraazole

The title compound was prepared using the procedure described in Example 28A except using 4-(chloromethyl)-5-methyl-1H-imidazole hydrochloride instead of 3-(chloromethyl)-2-methylpyridine hydrochloride. mp 151-153° C.; MS (ESI⁺) m/z 310 (M+H)⁺; ¹H NMR (DMSO-d₆) δ 11.95 (brs, 1H), 7.91 (dd, 1H, J=2.0, 7.5 Hz), 7.59 (dd, 1H, J=2.0, 7.6 Hz), 7.53 (t, 1H, J=7.6 Hz), 7.39 (s, 1H), 5.41 (s, 2H), 1.89 (s, 3H); Anal. calcd for C₁₂H₁₀Cl₂N₆: C, 46.62; H, 3.26; N, 27.18. Found: C, 46.65; H, 3.20; N, 26.89.

### Example 36

### 5-(2,3-dichlorophenyl)-1-[(2,4-dimethyl-1,3-thiazol-5-yl)methyl]-1H-tetraazole

The title compound was prepared using the procedure described in Example 28A except using 5-(chloromethyl)-2,4-dimethyl-1,3-thiazole hydrochloride instead of 3-(chloromethyl)-2-methylpyridine hydrochloride. mp 174-175° C.; MS (ESI⁺) m/z 341 (M+H)⁺; ¹H NMR (DMSO-d₆) δ 7.98 (dd, 1H, J=3.1, 6.8 Hz), 7.63 (dd, 1H, J=4.5, 7.6 Hz), 7.61 (t, 1H, J=7.5 Hz), 5.73 (s, 2H), 2.51 (s, 3H), 2.00 (s, 3H); Anal. calcd for C₁₃H₁₁Cl₂N₅S: C, 45.89; H, 3.26; N, 20.58. Found: C, 45.86; H, 3.13; N, 20.40.

### Example 37

### 5-(2,3-dichlorophenyl)-1-[(1-methyl-1H-imidazol-5-yl)methyl]-1H-tetraazole

The title compound was prepared using the procedure described in Example 28A except using 5-(chloromethyl)-1-methyl-1H-imidazole hydrochloride instead of 3-(chloromethyl)-2-methylpyridine hydrochloride. mp 166-168° C.; MS (ESI⁺) m/z 310 (M+H)⁺; ¹H NMR (DMSO-d₆) δ 7.96 (dd, 1H, J=1.7, 7.8 Hz), 7.67 (s, 1H), 7.64 (dd, 1H, J=1.7, 7.8 Hz), 7.61 (t, 1H, J=7.8 Hz), 6.58 (s, 1H), 5.68 (s, 2H), 3.51 (s, 3H); Anal. calcd for C₁₂H₁₀Cl₂N₆: C, 46.62; H, 3.26; N, 27.18. Found: C, 46.23; H, 3.13; N, 26.84.

### Example 38

### 5-(2,3-dichlorophenyl)-1-[(3,5-dimethylisoxazol-4-yl)methyl]-1H-tetraazole

The title compound was prepared using the procedure described in Example 28A except using 4-(chloromethyl)-3,5-dimethyl-isoxazole instead of 3-(chloromethyl)-2-methylpyridine hydrochloride. mp 176-178° C.; MS (ESI⁺) m/z 325 (M+H)⁺; ¹H NMR (DMSO-d₆) δ 7.96 (dd, 1H, J=1.7, 7.8 Hz), 7.70 (dd, 1H, J=1.7, 7.5 Hz), 7.62 (t, 1H, J=7.8 Hz), 5.44 (s, 2H), 2.04 (s, 3H), 1.96 (s, 3H); Anal. calcd for C₁₃H₁₁Cl₂N₅O: C, 48.17; H, 3.42; N, 21.60. Found: C, 47.92; H, 3.42; N, 21.47.

### Example 39

### 3-{[5-(2,5-dichlorophenyl)-1H-tetraazol-1-yl]methyl}pyridine hydrochloride

### Example 39A

### 3-{[5-(2,5-dichlorophenyl)-1H-tetraazol-1-yl]methyl}pyridine

The title compound was prepared using the procedure described in Example 28A except using 5-(2,5-dichlorophenyl)-1H-tetraazole (purchased from Lancaster) and 3-(bromomethyl)pyridine hydrobromide instead of 5-(2,3-dichlorophenyl)-1H-tetraazole and 3-(chloromethyl)-2-methylpyridine hydrochloride. mp 166-168° C.; MS (ESI⁺) m/z 307 (M+H)⁺; ¹H NMR (DMSO-d₆) δ 8.51 (dd, 1H, J=1.4, 4.8 Hz), 8.35 (d, 1H, J=2.0 Hz), 7.87 (d, 1H, J=2.7 Hz), 7.76 (dd, 1H, J=2.7, 8.7 Hz), 7.62 (d, 1H, J=8.2 Hz), 7.57 (td, 1H, J=2.0, 7.8 Hz), 7.34 (ddd, 1H, J=0.7, 4.8, 8.1 Hz), 5.65 (s, 2H); Anal. calcd for C₁₃H₉Cl₂N₅: C, 51.00; H, 2.96; N, 22.88. Found: C, 50.93; H, 2.83; N, 22.77.

### Example 39B

### 3-{[5-(2,5-dichlorophenyl)-1H-tetraazol-1-yl]methyl}pyridine hydrochloride

The title compound was prepared using the procedure described in Example 28B except using 3-{[5-(2,5-dichlorophenyl)-1H-tetraazol-1-yl]methyl}pyridine instead of 5-(2,3-dichlorophenyl)-1-[(2-methylpyridin-3-yl)methyl]-1H-tetraazole. mp 200-201° C.; MS (ESI⁺) m/z 307 (M+H)⁺; ¹H NMR (DMSO-d₆) δ 8.64 (dd, 1H, J=1.7, 5.1 Hz), 8.51 (d, 1H, J=1.7 Hz), 7.89 (d, 1H, J=2.7), 7.82 (m, 1H), 7.79 (dd, 1H, J=2.7, 8.8 Hz), 7.74 (t, 1H, J=9.2 Hz), 7.58 (dd, 1H, J=5.1, 8.1 Hz), 5.71 (s, 2H); Anal. calcd for C₁₃H₁₀Cl₃N₅: C, 45.57; H, 2.94; N, 20.44. Found: C, 45.78; H, 2.99; N, 20.62.

### Example 40

### 3-{[5-(2,5-dichlorophenyl)-1H-tetraazol-1-yl]methyl}-4-methylpyridine

The title compound was prepared using the procedure described in Example 28A except using 5-(2,5-dichlorophenyl)-1H-tetraazole and 3-(chloromethyl)-4-methylpyridine hydrochloride instead of 5-(2,3-dichlorophenyl)-1H-tetraazole and 3-(chloromethyl)-2-methylpyridine hydrochloride. mp 169-171° C.; MS (ESI⁺) m/z 321 (M+H)⁺; ¹H NMR (DMSO-d₆) δ 8.35 (d, 1H, J=2.0 Hz), 8.03 (s, 1H), 7.87 (d, 1H, J=2.7 Hz), 7.74 (dd, 1H, J=2.7, 8.6 Hz), 7.66 (d, 1H, J=8.8 Hz), 7.20 (d, 1H, J=5.1 Hz), 5.69 (s, 2H), 2.11 (s, 3H).

### Example 41

### 3-{[5-(3,4-dichlorophenyl)-1H-tetraazol-1-yl]methyl}pyridine

The title compound was prepared using the procedure described in Example 28A except using 5-(3,4-dichlorophenyl)-1H-tetraazole (purchased from Lancaster) and 3-(bromomethyl)pyridine hydrobromide instead of 5-(2,3-dichlorophenyl)-1H-tetraazole and 3-(chloromethyl)-2-methylpyridine hydrochloride. mp 166-168° C.; MS (ESI⁺) m/z 307 (M+H)⁺; ¹H NMR (DMSO-d₆) δ 8.53 (dd, 1H, J=1.7, 4.8 Hz), 8.45 (d, 1H, J=2.0 Hz), 8.05 (d, 1H, J=2.0 Hz), 7.88 (d, 1H, J=8.5 Hz), 7.75 (dd, 1H, J=2.0, 8.5 Hz), 7.58 (td, 1H, J=1.7, 7.8 Hz), 7.38 (ddd, 1H, J=1.0, 4.7, 8.1 Hz), 5.84 (s, 2H).

### Example 42

### 3-{[5-(3,4-dichlorophenyl)-1H-tetraazol-1-yl]methyl}-4-methylpyridine

The title compound was prepared using the procedure described in Example 28A except using 5-(3,4-dichlorophenyl)-1H-tetraazole and 3-(chloromethyl)-4-methylpyridine hydrochloride instead of 5-(2,3-dichlorophenyl)-1H-tetraazole and 3-(chloromethyl)-2-methylpyridine hydrochloride. mp 167-169° C.; MS (ESI⁺) m/z 321 (M+H)⁺; ¹H NMR (DMSO-d₆) δ 8.38 (d, 1H, J=5.1 Hz), 8.18 (s, 1H), 8.10 (d, 1H, J=2.0 Hz), 7.88 (d, 1H, J=8.5 Hz), 7.78 (dd, 1H, J=2.1, 8.5 Hz), 7.23 (d, 1H, J=4.8 Hz), 5.83 (s, 2H), 2.13 (s, 3H).

### Example 43

### 3-{[5-(3,4-dichlorophenyl)-1H-tetraazol-1-yl]methyl}-2-methylpyridine

The title compound was prepared using the procedure described in Example 28A except using 5-(3,4-dichlorophenyl)-1H-tetraazole instead of 5-(2,3-dichlorophenyl)-1H-tetraazole. mp 176-178° C.; MS (ESI⁺) m/z 321 (M+H)⁺; ¹H NMR (DMSO-d₆) δ 8.38 (dd, 1H, J=1.7, 4.8 Hz), 8.05 (d, 1H, J=2.0 Hz), 7.88 (d, 1H, J=8.5 Hz), 7.74 (dd, 1H, J=2.0, 8.5 Hz), 7.30 (dd, 1H, J=1.7, 7.8 Hz), 7.16 (dd, 1H, J=4.7, 7.8 Hz), 5.84 (s, 2H), 2.35 (s, 3H).

### Example 44

### 3-{[5-(2,5-dichlorophenyl)-1H-tetraazol-1-yl]methyl}-2-methylpyridine

The title compound was prepared using the procedure described in Example 28A except using 5-(2,5-dichlorophenyl)-1H-tetraazole instead of 5-(2,3-dichlorophenyl)-1H-tetraazole. mp 172-174° C.; MS (ESI⁺) m/z 321 (M+H)⁺; ¹H NMR (DMSO-d₆) δ 8.36 (dd, 1H, J=1.7, 4.8 Hz), 7.86 (d, 1H, J=2.0 Hz), 7.74 (dd, 1H, J=2.7, 8.7 Hz), 7.68 (d, 1H, J=8.8 Hz), 7.24 (dd, 1H, J=1.7, 7.8 Hz), 7.10 (dd, 1H, J=4.8, 7.8 Hz), 5.68 (s, 2H), 2.28 (s, 3H); Anal. calcd for C₁₄H₁₁Cl₂N₅; C, 52.52; H, 3.46; N, 21.87. Found: C, 52.43; H, 3.24; N, 21.53.

### Example 45

### 2-{[5-(2,3-dichlorophenyl)-1H-tetraazol-1-yl]methyl}phenyl acetate

The title compound was prepared using the procedure described in Example 28A except using 2-(chloromethyl)phenyl acetate instead of 3-(chloromethyl)-2-methylpyridine hydrochloride. mp 195-196° C.; MS (ESI⁺) m/z 364 (M+H)⁺; ¹H NMR (DMSO-d₆) δ 7.91 (dd, 1H, J=1.7, 7.6 Hz), 7.50 (t, 1H, J=7.8 Hz), 7.45 (dd, 1H, J=2.0, 7.8 Hz), 7.35 (dt, 1H, J=1.7, 7.5 Hz), 7.10 (m, 2H), 6.96 (dd, 1H, J=1.7, 8.0 Hz), 5.55 (s, 2H), 2.14 (s, 3H).

### Example 47

### 3-{2-[5-(2,3-dichlorophenyl)-1H-tetraazol-1-yl]ethyl}pyridine

The title compound was prepared using the procedure described in Example 28A except using 3-(2-chloroethyl)pyridine hydrochloride instead of 3-(chloromethyl)-2-methylpyridine hydrochloride.

The hydrochloride of the title compound was prepared as described in Example 28B. mp 112-113° C.; MS (ESI⁺) m/z 321 (M+H)⁺; ¹H NMR (DMSO-d₆) δ 8.70 (m, 2H), 8.22 (d, 1H, J=8.5 Hz), 7.87 (dd, 1H, J=1.4, 7.1 Hz), 7.80 (m, 2H), 7.55 (t, 1H, J=7.8 Hz), 5.20 (t, 2H, J=6.8 Hz), 3.40 (t, 2H, J=6.8 Hz); Anal. calcd for C₁₄H₁₂Cl₃N₅ 0.3H₂O: C, 46.25; H, 3.51; N, 19.34. Found: C, 46.60; H, 3.26; N, 18.97.

### Example 48

### 5-(2,3-dichlorophenyl)-1-(2-phenylethyl)-1H-tetraazole

The title compound was prepared using the procedure described in Example 28A except using 2-chloroethylbenzene instead of 3-(chloromethyl)-2-methylpyridine hydrochloride. MS (ESI⁺) m/z 320 (M+H)⁺; ¹H NMR (CDCl₃) δ 7.63 (dd, 1H, J=1.7, 8.1 Hz), 7.20 (m, 3H), 7.16 (t, 1H, J=7.8 Hz), 6.84 (dd, 2H, J=1.4, 7.5 Hz), 6.54 (dd, 1H, J=1.4, 7.5 Hz), 4.42 (t, 2H, J=6.8 Hz), 3.21 (t, 2H, J=6.8 Hz).

### Example 49

### 5-(2,3-dichlorophenyl)-1-(3-phenylpropyl)-1H-tetraazole

The title compound was prepared using the procedure described in Example 28A except using 3-chloropropylbenzene instead of 3-(chloromethyl)-2-methylpyridine hydrochloride. MS (ESI⁺) m/z 334 (M+H)⁺; ¹H NMR (CDCl₃) δ 7.85 (dd, 1H, J=1.7, 7.8 Hz), 7.61 (dd, 1H, J=1.7, 8.1 Hz), 7.20 (m, 5H), 7.07 (dd, 1H, J=1.7, 8.5 Hz), 4.71 (t, 2H, J=7.1 Hz), 2.73 (t, 2H, J=7.1 Hz), 2.42 (m, 2H).

### Example 50

### 1-benzyl-5-(5-chlorothien-2-yl)-1H-tetraazole

The title compound was prepared using the procedure described in Example 28A except using 5-(5-chlorothien-2-yl)-1H-tetraazole (purchased from Lancaster) and benzyl bromide instead of 5-(2,3-dichlorophenyl)-1H-tetraazole and 3-(chloromethyl)-2-methylpyridine hydrochloride. mp 176-177° C.; MS (ESI⁺) m/z 278 (M+H)⁺; ¹H NMR (DMSO-d₆) δ 7.61 (d, 1H, J=2.1 Hz), 7.36 (m, 3H), 7.34 (d, 1H, J=1.7 Hz), 7.19 (m, 2H), 5.94 (s, 2H); Anal. calcd for C₁₂H₉ClN₄S: C, 52.08; H, 3.28; N, 20.24. Found: C, 52.15; H, 3.15; N, 20.45.

### Example 51

### 1-benzyl-5-(5-bromothien-2-yl)-1H-tetraazole

The title compound was prepared using the procedure described in Example 28A except using 5-(5-bromothien-2-yl)-1H-tetraazole (purchased from Lancaster) and benzyl bromide instead of 5-(2,3-dichlorophenyl)-1H-tetraazole and 3-(chloromethyl)-2-methylpyridine hydrochloride. mp 196-197° C.; MS (ESI⁺) m/z 322 (M+H)⁺; ¹H NMR (DMSO-d₆) δ 7.57 (d, 1H, J=4.1 Hz), 7.43 (d, 1H, J=4.1 Hz), 7.38 (m, 3H), 7.19 (m, 2H), 5.93 (s, 2H); Anal. calcd for C₁₂H₉BrN₄S: C, 44.87; H, 2.82; N, 17.44. Found: C, 44.89; H, 2.63; N, 17.57.

### Example 52

### 3-[5-(2,3-dichlorophenyl-1H-tetraazol-1-yl]pyridine

2,3-Dichloro-N-pyridin-3-ylbenzamide (300 mg, 1.123 mmol), triethylamine (126 mg, 1.24 mmol), and DMF (23 mg, 0.315 mmol) were combined in acetonitrile (10 mL). After stirring for 60 minutes, the mixture was treated with thionyl chloride (174 mg, 0.623 mmol) keeping the reaction temperature below 25° C. The reaction mixture was stirred for 5 hours at room temperature, cooled to 10° C., treated with triethylamine (341 mg, 3.37 mmol), treated with sodium azide (160 mg, 2.46 mmol), and treated with tetrabutylammonium bromide (50 mg, 0.155 mmol). The mixture was stirred for 2 hours at 10° C. and then for 24 hours at room temperature. The mixture was treated with brine (15 mL) and extracted with dichloromethane (2×30 mL). The organic layer was washed with saturated aqueous NaHCO₃ solution (15 mL) and brine (15 mL). The organic portion was dried (Na₂SO₄), filtered, and the filtrate was concentrated under reduced pressure. The residue was purified by flash chromatography (elution with 2% ethanol, 49% ethyl acetate, 49% hexane) to provide the title compound. mp 124-126° C.; MS (ESI⁺) m/z 293 (M+H)⁺; ¹H NMR (DMSO-d₆) δ 8.73 (dd, 1H, J=1.7, 4.9 Hz), 8.68 (d, 1H, J=2.7 Hz), 7.95 (ddd, 1H, J=1.7, 2.5, 8.2 Hz), 7.90 (dd, 1H, J=1.7, 8.2 Hz), 7.78 (dd, 1H, J=1.3, 7.8 Hz), 7.61 (ddd, 1H, J=0.7, 4.7, 8.1 Hz), 7.57 (t, 1H, J=8.2 Hz); Anal. calcd for C₁₂H₇Cl₂N₅0.15 EtOAc: C, 49.56; H, 2.71; N, 22.94. Found: C, 49.27; H, 2.54; N, 22.70.

### Example 53

### 5-[2-fluoro-3-(trifluoromethyl)phenyl]-1-(2-methylbenzyl)-1H-tetraazole

### Example 53A

### 5-[2-fluoro-3-(trifluoromethyl)phenyl]-1H-tetraazole

2-Fluoro-3-(trifluoromethyl)benzonitrile (715 μL, 946 mg, 5.00 mmol) in toluene (2.5 mL) was treated with a solution of trimethylaluminum in toluene (2.50 mL of 2.0M, 5.00 mmol) dropwise followed by addition of neat azidotrimethylsilane (730 μL, 634 mg, 5.50 mmol) dropwise. The reaction mixture was heated at reflux overnight. After cooling to room temperature, the reaction mixture was diluted with ethyl acetate (20 mL), cooled in an ice bath, and treated with 1N HCl (10 mL) dropwise. The mixture was stirred vigorously at 0° C. for 1 hour and extracted with ethyl acetate (3×15 mL). The organic extracts were combined, dried over magnesium sulfate, filtered, and the filtrate was concentrated under reduced pressure. The residue was recrystallized from ethyl acetate/hexanes. ¹H NMR (CDCl₃) δ 12.65 (br s), 8.63 (dd, 1H), 7.87 (dd, 1H), 7.54 (dd, 1H); MS (ESI) calcd for C₈H₄F₄N₄: 232.0372: Found: 231.0 (M-H).

### Example 53B

### 5-[2-fluoro-3-(trifluoromethyl)phenyl]-1-(2-methylbenzyl)-1H-tetraazole

5-[2-Fluoro-3-(trifluoromethyl)phenyl]-1H-tetraazole (232 mg, 1.00 mmol) in anhydrous acetonitrile (8 mL) was treated with triethylamine (196 μL, 142 mg, 1.40 mmol), stirred at room temperature for 10 minutes, and treated with 1-(bromomethyl)-2-methylbenzene (161 μL, 222 mg, 1.20 mmol). After stirring at room temperature for 16 hours, the mixture was treated with water (10 mL) and extracted with dichloromethane (3×10 mL). The organic extracts were combined, dried over magnesium sulfate, filtered, and the filtrate was concentrated under reduced pressure. The residue was purified by flash chromatography (silica gel, 20% ethyl acetate in hexanes). ¹H NMR (CDCl₃) δ 7.81-7.75 (m, 1H), 7.58-7.53 (m, 1H), 7.31 (dd, J=7.8, 7.8 Hz, 1H), 7.17-7.06 (m, 2H), 7.01-6.96 (m, 1H), 6.83 (d, J=7.8 Hz, 1H), 5.63 (s, 2H), 2.09 (s, 3H); MS (ESI) calcd for C₁₆H₁₂F₄N₄: 336.0998: Found: 337.05 (M+H)⁺; Anal calcd for C₁₆H₁₂F₄N₄: C, 57.14%; H, 3.60%; N, 16.66%. Found: C, 57.61%; H, 3.78%; N, 16.30%.

### Example 54

### 1-(2-methylbenzyl)-5-(2-methylphenyl)-1H-tetraazole

The title compound was prepared using the procedure described in Example 53B except using 5-(2-methylphenyl)-1H-tetraazole instead of 5-[2-fluoro-3-(trifluoromethyl)phenyl]-1H-tetraazole. ¹H NMR (CDCl₃) δ 7.47-7.42 (m, 1H), 7.31-7.25 (m, 2H), 7.21-7.10 (m, 3H) 7.04 (dd, J=7.5 Hz, 1H), 6.72 (d, J=7.1 Hz, 1H), 5.41 (s, 2H), 2.13 (s, 3H), 1.97 (s, 3H). MS (ESI) calcd for C₁₆H₁₆N₄: 264.1375. Found: 265.06 (M+H)⁺; Anal calcd for C₁₆H₁₆N₄: C, 72.70%; H, 6.10%; N, 21.20%. Found: C, 72.59%; H, 6.12%; N, 21.20%.

### Example 55

### 5-(2,3-dimethylphenyl)-1-(2-methylbenzyl)-1H-tetraazole

### Example 55A

### 5-(2,3-dimethylphenyl)-1H-tetraazole

The title compound was prepared using the procedure described in Example 53A except using 2,3-dimethylbenzonitrile instead of 2-fluoro-3-(trifluoromethyl)benzonitrile. ¹H NMR (CDCl₃) δ 12.41 (br s, 1H), 7.37-7.34 (m, 2H), 7.22 (d, 1H), 2.38 (s, 6H); MS (ESI) calcd for C₉H₁₀N₄: 174.0905: Found: 173.0 (M-H).

### Example 55B

### 5-(2,3-dimethylphenyl)-1-(2-methylbenzyl)-1H-tetraazole

The title compound was prepared using the procedure described in Example 53B except using 5-(2,3-dimethylphenyl)-1H-tetraazole instead of 5-[2-fluoro-3-(trifluoromethyl)phenyl]-1H-tetraazole. ¹H NMR (CDCl₃) δ 7.32 (d, J=7.4 Hz, 1H), 7.21-7.15 (m, 2H), 7.10 (d, J=7.4 Hz, 1H), 7.04-6.97 (m, 2H), 6.68 (d, J=7.8 Hz, 1H), 5.38 (s, 2H), 2.27 (s, 3H), 2.12 (s, 3H), 1.76 (s, 3H); MS (ESI) calcd for C₁₇H₁₈N₄: 278.1531: Found: 279.05 (M+H)⁺; Anal calcd for C₁₇H₁₈N₄: C, 73.35%; H, 6.52%; N, 20.13%. Found: C, 73.37%; H, 6.45%; N, 20.17%.

### Example 56

### 5-(2,3-difluorophenyl)-1-(2-methylbenzyl)-1H-tetraazole

### Example 56A

### 5-(2,3-difluorophenyl)-1H-tetraazole

The title compound was prepared using the procedure described in Example 53A except using 2,3-difluorobenzonitrile instead of 2-fluoro-3-(trifluoromethyl)benzonitrile. ¹H NMR (CDCl₃) δ 12.67 (br s, 1H), 8.19-8.14 (m, 1H), 7.47-7.33 (m, 2H); MS (ESI) calcd for C₇H₄F₂N₄: 182.0404: Found: 181.0 (M-H).

### Example 56B

### 5-(2,3-difluorophenyl)-1-(2-methylbenzyl)-1H-tetraazole

The title compound was prepared using the procedure described in Example 53B except using 5-(2,3-difluorophenyl)-1H-tetraazole instead of 5-[2-fluoro-3-(trifluoromethyl)phenyl]-1H-tetraazole. ¹H NMR (CDCl₃) δ 7.42-7.33 (m, 1H), 7.23-7.10 (m, 4H), 7.07-7.01 (m, 1H), 6.82 (d, J=7.5 Hz, 1H), 5.58 (s, 2H), 2.16 (s, 3H); MS (ESI) calcd for C₁₅H₁₂F₂N₄: 286.1030: Found: 286.99 (M+H)⁺; Anal calcd for C₁₅H₁₂F₂N₄: C, 62.93%; H, 4.22%; N, 19.57%. Found: C, 63.04%; H, 4.20%; N, 19.62%.

### Example 57

### 3-{[5-(2,3-difluorophenyl)-1H-tetraazol-1-yl]methyl}pyridine

The title compound was prepared using the procedure described in Example 9 except using 5-(2,3-difluorophenyl)-1H-tetraazole and 3-bromomethylpyridine hydrobromide instead of 5-(2,3-dichlorophenyl)-1H-tetraazole and 4-bromomethylpyridine hydrobromide. ¹H NMR (CDCl₃) δ 8.57 (br s, 1H), 8.39 (br s, 1H), 7.54-7.50 (m, 1H), 7.48-7.40 (m, 1H), 7.31-7.21 (m, 3H), 5.58 (s, 2H); MS (ESI) calcd for C₁₃H₉F₂N₅: 273.0826: Found: 273.97 (M+H)⁺; Anal calcd for C₁₃H₉F₂N₅: C, 57.14%; H, 3.32%; N, 25.63%. Found: C, 57.08%; H, 3.30%; N, 25.39%.

### Example 58

### 3-({5-[2-fluoro-3-(trifluoromethyl)phenyl]-1H-tetraazol-1-yl}methyl)pyridine

The title compound was prepared using the procedure described in Example 9 except using 5-[2-fluoro-3-(trifluoromethyl)phenyl]-1H-tetraazole and 3-bromomethylpyridine hydrobromide instead of 5-(2,3-dichlorophenyl)-1H-tetraazole and 4-bromomethylpyridine hydrobromide. ¹H NMR (CDCl₃) δ 8.57 (br s, 1H), 8.39 (br s, 1H), 7.90-7.85 (m, 1H), 7.73-7.68 (m, 1H), 7.56 (d, J=7.8 Hz, 1H), 7.46-7.41 (m, 1H), 7.30 (br s, 1H), 5.61 (s, 2H); MS (ESI) calcd for C₁₄H₉F₄N₅: 323.0794: Found: 323.97 (M+H)⁺; Anal calcd for C₁₄H₉F₄N₅: C, 52.02%; H, 2.81%; N, 21.67%. Found: C, 51.86%; H, 2.75%; N, 21.58%.

### Example 59

### 1-benzyl-5-(2,6-dichlorophenyl)-1H-tetraazole

The title compound was prepared using the procedure described in Example 53B except using 5-(2,6-dichlorophenyl)-1H-tetraazole (purchased from Lancaster) and benzylbromide instead of 5-[2-fluoro-3-(trifluoromethyl)phenyl]-1H-tetraazole and 1-(bromomethyl)-2-methylbenzene. ¹H NMR (CDCl₃) δ 7.44-7.38 (m, 3H), 7.28-7.20 (m, 3H), 7.06-7.03 (m, 2H), 5.42 (s, 2H); MS (ESI) calcd for C₁₄H₁₀Cl₂N₄: 304.0283. Found: 304.95 (M+H)⁺; Anal calcd for C₁₄H₁₀Cl₂N₄: C, 55.10%; H, 3.30%; N, 18.36%. Found: C, 55.27%; H, 3.31%; N, 18.75

### Example 60

### 5-(2,6-dichlorophenyl)-1-[2-(trifluoromethyl)benzyl]-1H-tetraazole

The title compound was prepared using the procedure described in Example 53B except using 5-(2,6-dichlorophenyl)-1H-tetraazole and 1-(bromomethyl)-2-(trifluoromethyl)benzene instead of 5-[2-fluoro-3-(trifluoromethyl)phenyl]-1H-tetraazole and 1-(bromomethyl)-2-methylbenzene. ¹H NMR (CDCl₃) δ 7.62 (d, J=8.5 Hz, 1H), 7.50 (m, 5H), 7.14 (d, J=7.5 Hz, 1H), 5.65 (s, 2H); MS (ESI) calcd for C₁₅H₉Cl₂F₃N₄: 372.0156. Found: 372.94 M+H)⁺; Anal calcd for C₁₅H₉Cl₂F₃N₄: C, 48.28%; H, 2.43%; N, 15.01%. Found: C, 48.01%; H, 2.36%; N, 15.43%.

### Example 61

### 1-benzyl-5-(2,3-difluorophenyl)-1H-tetraazole

The title compound was prepared using the procedure described in Example 53B except using 5-(2,3-difluorophenyl)-1H-tetraazole and benzyl bromide instead of 5-[2-fluoro-3-(trifluoromethyl)phenyl]-1H-tetraazole and 1-(bromomethyl)-2-methylbenzene. ¹H NMR (CDCl₃) δ 7.44-7.34 (m, 1H), 7.29-7.14 (m, 5H), 7.08-7.05 (m, 2H), 5.57 (s, 2H); MS (ESI) calcd for C₁₄H₁₀F₂N₄: 272.0874: Found: 273.00; Anal calcd for C₁₄H₁₀F₂N₄: C, 61.76; H, 3.70; N, 20.58%. Found: C, 61.41%; H, 3.59%; N, 20.42%.

### Example 62

### 5-(2,3-difluorophenyl)-1-[2-(trifluoromethyl)benzyl]-1H-tetraazole

The title compound was prepared using the procedure described in Example 53B except using 5-(2,3-difluorophenyl)-1H-tetraazole and 1-(bromomethyl)-2-(trifluoromethyl)benzene instead of 5-[2-fluoro-3-(trifluoromethyl)phenyl]-1H-tetraazole and 1-(bromomethyl)-2-methylbenzene. ¹H NMR (CDCl₃) δ 7.69-7.64 (m, 1H), 7.55-7.35 (m, 3H), 7.27-7.20 (m, 2H) 7.07 (d, J=7.4 Hz, 1H), 5.75 (s, 2H); MS (ESI) calcd for C₁₅H₉F₅N₄: 340.0747: Found: 340.97 (M+H)⁺; Anal calcd for C₁₅H₉F₅N₄: C, 52.95%; H, 2.67%; N, 16.47%. Found: C, 52.82%; H, 2.67%; N, 16.26%.

### Example 63

### 1-benzyl-5-(2,3-dimethylphenyl)-1H-tetraazole

The title compound was prepared using the procedure described in Example 53B except using 5-(2,3-dimethylphenyl)-1H-tetraazole and benzyl bromide instead of 5-[2-fluoro-3-(trifluoromethyl)phenyl]-1H-tetraazole and 1-(bromomethyl)-2-methylbenzene. ¹H NMR (CDCl₃) δ 7.34 (d, J=7.8 Hz, 1H), 7.30-7.17 (m, 4H), 7.02-6.96 (m, 3H), 5.35 (s, 2H), 2.30 (s, 3H), 1.79 (s, 3H); MS (ESI) calcd for C₁₆H₁₆N₄: 264.1375. Found: 265.06 (M+H)⁺; Anal calcd for C₁₆H₁₆N₄: C, 72.70%; H, 6.10%; N, 21.20%. Found: C, 72.63%; H, 5.91%; N, 21.28%.

### Example 64

### 5-(2,3-dimethylphenyl)-1-[2-(trifluoromethyl)benzyl]-1H-tetraazole

The title compound was prepared using the procedure described in Example 53B except using 5-(2,3-dimethylphenyl)-1H-tetraazole and 1-(bromomethyl)-2-(trifluoromethyl)benzene instead of 5-[2-fluoro-3-(trifluoromethyl)phenyl]-1H-tetraazole and 1-(bromomethyl)-2-methylbenzene. ¹H NMR (CDCl₃) δ 7.65 (d, J=7.4 Hz, 1H), 7.53-7.40 (m, 2H), 7.30 (d, J=7.5 Hz, 1H), 7.13 (dd, J=7.6, 7.6 Hz, 1H), 6.96 (d, J=7.8 Hz, 1H), 6.91 (d, J=7.5 Hz, 1H), 5.60 (s, 2H), 2.30 (s, 3H), 1.99 (s, 3H); MS (ESI) calcd for C₁₇H₁₅F₃N₄: 332.1249: Found: 333.00 (M+H)⁺; Anal calcd for C₁₇H₁₅F₃N₄: C, 61.44%; H, 4.55%; N, 16.86%. Found: C, 61.45%; H, 4.52%; N, 16.77%.

### Example 65

### 5-(2,3-difluorophenyl)-1-(2-methylbenzyl)-1H-tetraazole

The title compound was prepared using the procedure described in Example 53B except using 5-(2,3-difluorophenyl)-1H-tetraazole instead of 5-[2-fluoro-3-(trifluoromethyl)phenyl]-1H-tetraazole. ¹H NMR (CDCl₃) δ 8.45-8.43 (m, 1H), 7.47-7.38 (m, 1H), 7.30-7.21 (m, 5H), 7.19 (d, J=8.2 Hz, 1H), 7.06 (d, J=7.8 Hz, 1H), 7.04 (d, J=7.8 Hz, 1H), 5.59 (s, 2H), 2.40 (s, 3H); MS (ESI) calcd for C₁₄H₁₁F₂N₅: 287.0983. Found: 288.00 (M+H)⁺; Anal calcd for C₁₄H₁₁F₂N₅: C, 58.53%; H, 3.86%; N, 24.38%. Found: C, 58.52%; H, 3.88%; N, 24.41%.

### Example 66

### 5-(3-chloro-2-fluorophenyl)-1-(2-methylbenzyl)-1H-tetraazole

### Example 66A

### 5-(3-chloro-2-fluorophenyl)-1H-tetraazole

The title compound was prepared using the procedure described in Example 53A except using 3-chloro-2-fluorobenzonitrile instead of 2-fluoro-3-(trifluoromethyl)benzonitrile. ¹H NMR (CDCl₃) δ 12.78 (br s, 1H), 8.31 (ddd, 1H), 7.65 (ddd, 1H), 7.36 (dd, 1H); MS (ESI) calcd for C₇H₄ClFN₄: 198.0109. Found: 196.9 (M-H).

### Example 66B

### 5-(3-chloro-2-fluorophenyl)-1-(2-methylbenzyl)-1H-tetraazole

The title compound was prepared using the procedure described in Example 53B except using 5-(3-chloro-2-fluorophenyl)-1H-tetraazole instead of 5-[2-fluoro-3-(trifluoromethyl)phenyl]-1H-tetraazole. ¹H NMR (CDCl₃) δ 7.62-7.56 (m, 1H), 7.31-7.25 (m, 1H), 7.20-7.00 (4H), 6.82 (d, J=7.5 Hz, 1H), 5.58 (s, 2H), 2.14 (s, 3H); MS (ESI) calcd for C₁₅H₁₂ClFN₄: 302.0735. Found: 302.96 (M+H)⁺; Anal calcd for C₁₅H₁₂ClFN₄: C, 59.51%; H, 4.00%; N, 18.51%. Found: C, 59.29%; H, 3.99%; N, 18.43%.

### Example 67

### 1-(2-methylbenzyl)-5-[2-(trifluoromethyl)phenyl]-1H-tetraazole

### Example 67A

### 5-[2-(trifluoromethyl)phenyl]-1H-tetraazole

The title compound was prepared using the procedure described in Example 53A except using 2-(trifluoromethyl)benzonitrile instead of 2-fluoro-3-(trifluoromethyl)benzonitrile. ¹H NMR (CDCl₃) δ 7 12.38 (br s, 1H), 8.02 (br d, 1H), 7.90 (dd, 1H), 7.77-7.72 (m, 2H); MS (ESI) calcd for C₈H₅F₃N₄: 214.0466. Found: 213.0 (M-H).

### Example 67B

### 1-(2-methylbenzyl)-5-[2-(trifluoromethyl)phenyl]-1H-tetraazole

The title compound was prepared using the procedure described in Example 53B except using 5-[2-(trifluoromethyl)phenyl]-1H-tetraazole instead of 5-[2-fluoro-3-(trifluoromethyl)phenyl]-1H-tetraazole. ¹H NMR (CDCl₃) δ 7.85-7.83 (m, 1H), 7.71-7.66 (m, 1H), 7.56-7.51 (m, 1H), 7.21-7.16 (m, 1H), 7.10 (d, J=7.5 Hz, 1H), 7.05-6.96 (m, 2H), 6.69 (d, J=7.8 Hz, 1H), 5.41 (s, 2H), 2.12 (s, 3H); MS (ESI) calcd for C₁₆H₁₃F₃N₄: 318.1092: Found: 319.02 (M+H)⁺; Anal calcd for C₁₆H₁₃F₃N₄: C, 60.37%; H, 4.12%; N, 17.60%. Found: C, 60.08%; H, 4.05%; N, 17.57%.

### Example 68

### 3-({5-[2-fluoro-3-(trifluoromethyl)phenyl]-1H-tetraazol-1-yl}methyl)-2-methylpyridine

The title compound was prepared using the procedure described in Example 53B except using 3-(bromomethyl)-2-methylpyridine instead of 1-(bromomethyl)-2-methylbenzene. ¹H NMR (CDCl₃) δ 8.41 (dd, J=7.8, 1.8 Hz, 1H), 7.87-7.81 (m, 1H), 7.69-7.64 (m, 1H), 7.43-7.37 (m, 1H), 7.23 (d, J=7.5 Hz, 1H), 7.02 (dd, J=7.5, 4.6 Hz, 1H), 5.63 (s, 2H), 2.34 (s, 3H); MS (ESI) calcd for C₁₅H₁₁F₄N₅: 337.0951: Found: 338.01; Anal calcd for C₁₅H₁₁F₄N₅: C, 53.42%; H, 3.29%; N, 20.76%. Found: C, 53.52%; H, 3.32%; N, 20.52%.

### Example 69

### 1-benzyl-5-[2-(trifluoromethyl)phenyl]-1H-tetraazole

The title compound was prepared using the procedure described in Example 53B except using 5-[2-(trifluoromethyl)phenyl]-1H-tetraazole and benzyl bromide instead of 5-[2-fluoro-3-(trifluoromethyl)phenyl]-1H-tetraazole and 1-(bromomethyl)-2-methylbenzene. ¹H NMR (CDCl₃) δ 7.89-7.86 (m, 1H), 7.74-7.69 (m, 1H), 7.61-7.56 (m, 1H), 7.34-7.23 (m, 3H), 7.08 (d, J=8.2 Hz, 1H), 7.02-6.99 (m, 2H), 5.37 (s, 2H); MS (ESI) calcd for C₁₅H₁₁F₃N₄: 304.0936: Found: 305.01; Anal calcd for C₁₅H₁₁F₃N₄: C, 59.21%; H, 3.64%; N, 18.41%. Found: C, 59.22%; H, 3.46%; N, 18.56%.

### Example 70

### 3-({5-[2-(trifluoromethyl)phenyl]-1H-tetraazol-1-yl}methyl)pyridine

The title compound was prepared using the procedure described in Example 9 except using 5-[2-(trifluoromethyl)phenyl]-1H-tetraazole and 3-bromomethylpyridine hydrobromide instead of 5-(2,3-dichlorophenyl)-1H-tetraazole and 4-bromomethylpyridine hydrobromide. ¹H NMR (CDCl₃) δ 78.58 (dd, J=4.7, 1.4 Hz, 1H), 8.22 (d, J=2.1 Hz, 1H), 7.91 (d, J=7.8 Hz, 1H), 7.77 (dd, J=7.5, 7.5 Hz, 1H), 7.67 (dd, J=7.5, 7.5 Hz, 1H), 7.56-7.51 (m, 1H), 7.30-7.25 (m, 1H), 7.18 (d, J=7.5 Hz, 1H), 5.39 (s, 2H); MS (ESI) calcd for C₁₄H₁₀F₃N₅: 305.0888: Found: 305.98; Anal calcd for C₁₄H₁₀F₃N₅: C, 55.08%; H, 3.30%; N, 22.94%. Found: C, 54.45%; 3.36%; N, 23.49%.

### Example 71

### 3-({5-[3-(trifluoromethyl)phenyl]-1H-tetraazol-1-yl}methyl)pyridine

### Example 71A

### 5-[3-(trifluoromethyl)phenyl]-1H-tetraazole

The title compound was prepared using the procedure described in Example 53A except using 3-(trifluoromethyl)benzonitrile instead of 2-fluoro-3-(trifluoromethyl)benzonitrile. ¹H NMR (CDCl₃) δ 13.22 (br s, 1H), 8.42-8.34 (m, 2H), 7.81-7.68 (m, 2H); MS (ESI) calcd for C₈H₅F₃N₄: 214.0466: Found: 213.0 (M-H).

### Example 71B

### 3-({5-[3-(trifluoromethyl)phenyl]-1H-tetraazol-1-yl}methyl)pyridine

The title compound was prepared using the procedure described in Example 9 except using 5-[3-(trifluoromethyl)phenyl]-1H-tetraazole and 3-bromomethylpyridine hydrobromide instead of 5-(2,3-dichlorophenyl)-1H-tetraazole and 4-bromomethylpyridine hydrobromide. ¹H NMR (CDCl₃) δ 8.65 (br s, 1H), 8.51 (br s, 1H), 7.88-7.85 (m, 2H), 7.78 (d, J=7.8 Hz, 1H), 7.72-7.67 (m, 1H), 7.58 (d, J=7.8 Hz, 1H), 7.36-7.32 (m, 1H), 5.66 (s, 2H); MS (ESI) calcd for C₁₄H₁₀F₃N₅: 305.0888: Found: 305.98; Anal calcd for C₁₄H₁₀F₃N₅: C, 55.08%; H, 3.03%; N, 22.94%. Found: C, 55.07%; H, 3.25%; N, 22.67%.

### Example 72

### 1-benzyl-5-(2-bromophenyl)-1H-tetraazole

### Example 72A

### 5-(2-bromophenyl)-1H-tetraazole

The title compound was prepared using the procedure described in Example 53A except using 2-bromobenzonitrile instead of 2-fluoro-3-(trifluoromethyl)benzonitrile. ¹H NMR (CDCl₃) δ 12.95 (br s, 1H), 8.33-8.31 (m, 1H), 7.76 (d, 1H), 7.55 (dd, 1H), 7.44 (ddd, 1H); MS (ESI) calcd for C₇H₅BrN₄: 223.9698: Found: 222.9 (M-H).

### Example 72B

### 1-benzyl-5-(2-bromophenyl)-1H-tetraazole

The title compound was prepared using the procedure described in Example 53B except using 5-(2-bromophenyl)-1H-tetraazole and benzyl bromide instead of 5-[2-fluoro-3-(trifluoromethyl)phenyl]-1H-tetraazole and 1-(bromomethyl)-2-methylbenzene. ¹H NMR (CDCl₃) δ 7.73 (dd, J=7.4, 1.4 Hz, 1H), 7.46-7.40 (m, 1H), 7.40-7.34 (m, 1H), 7.30-7.20 (m, 3H), 7.13 (dd, J=7.4-2.1 Hz, 1H), 7.02-6.99 (m, 2H), 5.46 (s, 2H); MS (ESI) calcd for C₁₄H₁₁BrN₄: 314.0167: Found: 316.83; Anal calcd for C₁₄H, BrN₄: C, 53.35%; H, 3.52%; N, 17.78%. Found: C, 53.50%; H, 3.36%; N, 17.88%.

### Example 73

### 1-benzyl-5-(3-bromophenyl)-1H-tetraazole

### Example 73A

### 5-(3-bromophenyl)-1H-tetraazole

The title compound was prepared using the procedure described in Example 53A except using 3-bromobenzonitrile instead of 2-fluoro-3-(trifluoromethyl)benzonitrile. ¹H NMR (CDCl₃) δ 13.25 (br s, 1H), 8.28 (br s, 1H), 8.07-8.04 (m, 1H), 7.67 (d, 1H), 7.42 (dd, 1H); MS (ESI) calcd for C₇H₅BrN₄: 223.9698: Found: 224.8 (M-H).

### Example 73B

### 1-benzyl-5-(3-bromophenyl)-1H-tetraazole

The title compound was prepared using the procedure described in Example 53B except using 5-(3-bromophenyl)-1H-tetraazole and benzyl bromide instead of 5-[2-fluoro-3-(trifluoromethyl)phenyl]-1H-tetraazole and 1-(bromomethyl)-2-methylbenzene. ¹H NMR (CDCl₃) δ 7.74-7.68 (m, 2H), 7.53-7.49 (m, 1H), 7.40-7.35 (4H), 7.19-7.16 (m, 2H), 5.62 (s, 2H); MS (ESI) calcd for C₁₄H₁₁BrN₄: 314.0167: Found: 314.93 (M+H)⁺; Anal calcd for C₁₄H₁₁BrN₄: C, 53.35%; H, 3.52%; N, 17.78%. Found: C, 53.62%; H, 3.42%; N, 17.97%.

### Example 74

### 1-benzyl-5-(4-bromo-2,3,5,6-tetrafluorophenyl)-1H-tetraazole

### Example 74A

### 5-(4-bromo-2,3,5,6-tetrafluorophenyl)-1H-tetraazole

The title compound was prepared using the procedure described in Example 53A except using 4-bromo-2,3,5,6-tetrafluorobenzonitrile instead of 2-fluoro-3-(trifluoromethyl)benzonitrile. ¹H NMR (CDCl₃) δ 13.4 (br s, 1H); MS (ESI) calcd for C₇H₁BrF₄N₄: 295.9321: Found: 296.8 (M+H).

### Example 74B

### 1-benzyl-5-(4-bromo-2,3,5,6-tetrafluorophenyl)-1H-tetraazole

The title compound was prepared using the procedure described in Example 53B except using 5-(4-bromo-2,3,5,6-tetrafluorophenyl)-1H-tetraazole and benzyl bromide instead of 5-[2-fluoro-3-(trifluoromethyl)phenyl]-1H-tetraazole and 1-(bromomethyl)-2-methylbenzene. ¹H NMR (CDCl₃) δ 7.34-7.25 (m, 3H), 7.07-7.02 (m, 2H), 5.57 (s, 2H); MS (ESI) calcd for C₁₄H₇BrF₄N₄: 385.9790: Found: 388.4 (M-H); Anal calcd for C₁₄H₇BrF₄N₄: C, 43.43%; H, 1.82%; N, 14.47%. Found: C, 43.57%; H, 1.72%; N, 14.42%.

### Example 75

### 1-benzyl-5-(3-iodophenyl)-1H-tetraazole

### Example 75A

### 5-(3-iodophenyl)-1H-tetraazole

The title compound was prepared using the procedure described in Example 53A except using 3-iodobenzonitrile instead of 2-fluoro-3-(trifluoromethyl)benzonitrile. ¹H NMR (CDCl₃) δ 13.2 (br s, 1H), 8.47 (br s, 1H), 8.07 (br d, 1H), 7.87 (d, 1H), 7.28 (dd, 1H); MS (ESI) calcd for C₇H₅₁N₄: 271.9559: Found: 270.9 (M-H).

### Example 75B

### 1-benzyl-5-(3-iodophenyl)-1H-tetraazole

The title compound was prepared using the procedure described in Example 53B except using 5-(3-iodophenyl)-1H-tetraazole and benzyl bromide instead of 5-[2-fluoro-3-(trifluoromethyl)phenyl]-1H-tetraazole and 1-(bromomethyl)-2-methylbenzene. ¹H NMR (CDCl₃) δ 7.92-7.86 (m, 2H), 7.57-7.53 (m, 1H), 7.40-7.35 (m, 3H), 7.26-7.16 (m, 3H), 5.61 (s, 2H); MS (ESI) calcd for C₁₄H₁₁IN₄: 362.0028: Found: 362.88 (M+H)⁺; Anal calcd for C₁₄H₁₁IN₄: C, 46.43%; H, 3.06%; N, 15.47%. Found: C, 46.38%; H, 2.80%; N, 15.49%.

### Example 76

### 1-benzyl-5-(3-bromo-4-fluorophenyl)-1H-tetraazole

### Example 76A

### 5-(3-bromo-4-fluorophenyl)-1H-tetraazole

The title compound was prepared using the procedure described in Example 53A except using 3-bromo-4-fluorobenzonitrile instead of 2-fluoro-3-(trifluoromethyl)benzonitrile. ¹H NMR (CDCl₃) δ 13.3 (br s, 1H), 8.38-8.36 (m, 1H), 7.30 (d, 1H); MS (ESI) calcd for C₇H₄BrFN₄: 241.9603: Found: 240.9 (M-H).

### Example 76B

### 1-benzyl-5-(3-bromo-4-fluorophenyl)-1H-tetraazole

The title compound was prepared using the procedure described in Example 53B except using 5-(3-bromo-4-fluorophenyl)-1H-tetraazole and benzyl bromide instead of 5-[2-fluoro-3-(trifluoromethyl)phenyl]-1H-tetraazole and 1-(bromomethyl)-2-methylbenzene. ¹H NMR (CDCl₃) δ 7.79 (dd, J=5.8, 2.4 Hz, 1H), 7.54-7.49 (m, 1H), 7.41-7.36 (m, 3H), 7.27-7.21 (m, 1H), 7.19-7.15 (m, 2H), 5.62 (s, 2H); MS (ESI) calcd for C₁₄H₁₀BrFN₄: 332.0073: Found: 334.8; Anal calcd for C₁₄H₁₀BrFN₄: C, 50.47%; H, 3.03%; N, 16.82%. Found: C, 50.81%; H, 3.05%; N, 16.89%.

### Example 77

### 5-(2,3-dichlorophenyl)-1-(thien-3-ylmethyl)-1H-tetraazole

Thien-3-ylmethanol (207 mg, 1.81 mmol) in THF (7 mL) was treated with 5-(2,3-dichlorophenyl)-1H-tetraazole (300 mg, 1.39 mmol), triphenylphosphine (475 mg, 1.81 mmol), and diethylazodicarboxylate (475 mg, 1.81 mmo). The solution was stirred at ambient temperature for 6 hours then partitioned between EtOAc (15 mL) and 10% HCl (10 mL). The organic phase was washed with brine (5 mL), dried (Na₂SO₄), filtered, and the filtrate was concentrated under reduced pressure. The residue was purified by reverse phase HPLC (gradient elution with 10-100% CH₃CN containing 0.01M aq. NH₄OAc) to provide the desired product (51 mg). MS (DCI/NH₃) m/z 311 (M+H)⁺; ¹H NMR (DMSO-d₆) δ 7.94 (dd, 1H, J=7.0, 2.8 Hz), 7.62-7.53 (m, 2H), 7.48 (dd, 1H, J=4.9, 2.8 Hz), 7.28-7.26 (m, 1H), 6.85 (dd, 1H, J=4.9, 1.2 Hz), 4.58 (s, 2H); Anal. calcd for C₁₂H₈Cl₂N₄S: C, 46.32; H, 2.59; N, 18.00. Found: C, 46.29; H, 2.57; N, 18.07.

### Example 78

### N-{[2-({5-[2-chloro-3-(trifluoromethyl)phenyl]-1H-tetraazol-1-yl}methyl)thien-3-yl]methyl}-N,N-dimethylamine

### Example 78A

### N-{[2-(chloromethyl)thien-3-yl]methyl}-N,N-dimethylamine

Thionyl chloride (1 mL) in methylene chloride (15 mL) was treated with {3-[(dimethylamino)methyl]thien-2-yl}methanol (Brown et al.; Eur. J. Med. Chem. Chim. Ther. (1990), 25(3); 217-226) (600 mg, 3.51 mmol) at room temperature, stirred for 3 hours, and then concentrated under reduced pressure to provide the title compound. MS (DCI⁺) m/z 190 (M+H)⁺.

### Example 78B

### N-{[2-(azidomethyl)thien-3-yl]methyl}-N,N-dimethylamine

N-{[2-(Chloromethyl)thien-3-yl]methyl}-N,N-dimethylamine (580 mg, 3.06 mmol) and sodium azide (596 mg, 9.18 mmol) were combined in ethanol (10 mL), stirred at 60° C. under nitrogen atmosphere for 2 days, and concentrated under reduced pressure. The residue was treated with dichloromethane, filtered, and the filtrate was concentrated under reduced pressure. The residue was purified by flash chromatography (elution with 50% ethyl acetate, 50% dichloromethane) to provide the title compound. MS (DCI⁺) m/z 197 (M+H)⁺.

### Example 78C

### N-{[2-(aminomethyl)thien-3-yl]methyl}-N,N-dimethylamine

N-{[2-(Azidomethyl)thien-3-yl]methyl}-N,N-dimethylamine (470 mg, 2,40 mmol) in anhydrous THF (20 mL) at 0° C. was treated with LiAlH₄. After stirring at 0° C. for 4 hours, the mixture was treated with aqueous ammonium chloride, filtered, and the filter cake was washed with MeOH/CH₂Cl₂ (1/10). The filtrate was concentrated under reduced pressure to provide the title compound. MS (DCI⁺) m/z 171 (M+H)⁺.

### Example 78D

### 2-chloro-N-({3-[(dimethylamino)methyl]thien-2-yl}methyl)-3-(trifluoromethyl)benzamide

The title compound was prepared using the procedure described in Example 85B except using N-{[2-(aminomethyl)thien-3-yl]methyl}-N,N-dimethylamine and 2-chloro-3-(trifluoromethyl)benzoic acid instead of 3-(aminomethyl)pyridine and 3-bromo-2-chlorobenzoic acid. MS (ESI⁺) m/z 377 (M+H)⁺.

### Example 78E

### 2-chloro-N-({3-[(dimethylamino)methyl]thien-2-yl}methyl)-3-(trifluoromethyl)benzenecarbothioamide

2-Chloro-N-({3-[(dimethylamino)methyl]thien-2-yl}methyl)-3-(trifluoromethyl)benzamide (100 mg, 0.27 mmol) and Lawesson's reagent (54 mg, 0.13 mmol) were combined in toluene (1.5 mL), refluxed for 4 hours, cooled to room temperature, and concentrated under reduced pressure. The residue was purified by flash chromatography, eluting with CH₂Cl₂/EtOAc (4/1) to provide the title compound. MS (ESI⁺) m/z 392 (M).

### Example 78F

### N-{[2-({5-[2-chloro-3-(trifluoromethyl)phenyl]-1H-tetraazol-1-yl}methyl)thien-3-yl]methyl}-N,N-dimethylamine

2-Chloro-N-({3-[(dimethylamino)methyl]thien-2-yl}methyl)-3-(trifluoromethyl)benzenecarbothioamide (50 mg, 0.13 mmol), mercuric acetate (41 mg, 0.13 mmol), and azidotrimethylsilane (0.123 mL, 0.91 mmol) were combined in anhydrous THF (3 mL), stirred at room temperature for 8 hours, filtered, filter cake was washed with dichloromethane, and the filtrate was concentrated under reduced pressure. The residue was purified by reverse phase HPLC to provide the title compound. MS (ESI⁺) m/z 402 (M+H)⁺; ¹H NMR (300 MHz, DMSO-d₆) δ 1.93 (s, 6H), 3.30 (s, 2H), 5.85 (s, 2H), 6.87 (d, J=5.1 Hz, 1H), 7.45 (d, J=4.8 Hz, 1H), 7.81 (t, J=8.1 Hz, 1H), 7.97 (dd, J=7.5, 1.4 Hz, 1H), 8.18 (dd, J=8.1, 1.4 Hz, 1H).

### Example 79

### 3-({5-[2-chloro-3-(trifluoromethyl)phenyl]-1H-tetraazol-1-yl}methyl)quinoline

### Example 79A

### 2-chloro-N-(quinolin-3-ylmethyl)-3-(trifluoromethyl)benzamide

The title compound was prepared using the procedure described in Example 85B except using quinolin-3-ylmethylamine and 2-chloro-3-(trifluoromethyl)benzoic acid instead of 3-(aminomethyl)pyridine and 3-bromo-2-chlorobenzoic acid. MS (ESI⁺) m/z 365 (M+H)⁺.

### Example 79B

### 2-chloro-N-(quinolin-3-ylmethyl)-3-(trifluoromethyl)benzenecarbothioamide

The title compound was prepared using the procedure described in Example 78E except using 2-chloro-N-(quinolin-3-ylmethyl)-3-(trifluoromethyl)benzamide instead of 2-chloro-N-({3-[(dimethylamino)methyl]thien-2-yl}methyl)-3-(trifluoromethyl)benzamide. MS (ESI⁺) m/z 380 (M).

### Example 79C

### 3-({5-[2-chloro-3-(trifluoromethyl)phenyl]-1H-tetraazol-1-yl}methyl)quinoline

2-Chloro-N-(quinolin-3-ylmethyl)-3-(trifluoromethyl)benzenecarbothioamide (48 mg, 0.13 mmol), mercuric acetate (50 mg, 0.16 mmol), and azidotrimethylsilane (0.18 mL, 1.26 mmol) were combined in anhydrous THF (2 mL), stirred at room temperature for 8 hours, filtered, the filter cake was washed with dichloromethane, and the filtrate was concentrated under reduced pressure. The residue was purified by flash chromatography (ethyl acetate:dichloromethane, 1:1) provide the title compound. MS (DCI⁺) m/z 390 (M+H)⁺; ¹H NMR (300 MHz, DMSO-d₆) δ 5.86 (s, 2H), 7.57-7.64 (m, 1H), 7.72-7.80 (m, 2H), 7.87 (dd, J=8.5, 1.0 Hz, 1H), 7.98 (d, J=7.8 Hz, 1H), 8.02-8.11 (m, 3H), 8.64 (d, J=2.4 Hz, 1H).

### Example 80

### 3-{[5-(3-chloro-2,4-difluorophenyl)-1H-tetraazol-1-yl]methyl}pyridine

### Example 80A

### 3-chloro-2,4-difluorobenzamide

The title compound was prepared using the procedure described in Example 91A except using 3-chloro-2,4-difluorobenzoic acid instead of 2-chloro-3-(trifluoromethyl)benzoic acid. ¹H NMR (DMSO-d₆) δ 7.88 (br s, 1H), 7.77 (br s, 1H), 7.68 (ddd, 1H), 7.39 (ddd, 1H); MS (ESI) calcd for C₇H₄ClF₂NO: 190.9941. Found: 189.9 (M-H).

### Example 80B

### 3-chloro-2,4-difluorobenzonitrile

The title compound was prepared using the procedure described in Example 91B except using 3-chloro-2,4-difluorobenzamide instead of 2-chloro-3-(trifluoromethyl)benzamide. ¹H NMR (CDCl₃) δ 7.57 (ddd, 1H), 7.13 (ddd, 1H).

### Example 80C

### 5-(3-chloro-2,4-difluorophenyl)-1H-tetraazole

The title compound was prepared using the procedure described in Example 53A except using 3-chloro-2,4-difluorobenzonitrile instead of 2-fluoro-3-(trifluoromethyl)benzonitrile. ¹H NMR (CDCl₃) δ 13.0 (br s, 1H), 8.32 (ddd, 1H), 7.26 (ddd, 1H); MS (ESI) calcd for C₇H₃ClF₂N₄: 216.0014. Found: 215.0 (M-H).

### Example 80D

### 3-{[5-(3-chloro-2,4-difluorophenyl)-1H-tetraazol-1-yl]methyl}pyridine

The title compound was prepared using the procedure described in Example 9 except using 5-(3-chloro-2,4-difluorophenyl)-1H-tetraazole and 3-bromomethylpyridine hydrobromide instead of 5-(2,3-dichlorophenyl)-1H-tetraazole and 4-bromomethylpyridine hydrobromide. MS (ESI+) m/z 308 (M+H)⁺; ¹H NMR (CDCl₃) δ 8.60 (br s, 1H), 8.43 (br s, 1H), 7.58 (d, 1H), 7.46-7.39 (m, 1H), 7.35-7.30 (m, 1H), 7.21-7.14 (m, 1H), 5.58 (s, 2H).

### Example 81

### 3-({5-[2-iodo-3-(trifluoromethyl)phenyl]-1H-tetraazol-1-yl}methyl)pyridine

### Example 81A

### 2-iodo-3-(trifluoromethyl)benzoic acid

The title compound was prepared using the procedure described in Example 86A except using 2-amino-3-(trifluoromethyl)benzoic acid instead of 3-amino-2-chloro-benzoic acid. ¹H NMR (DMSO-d₆) δ 13.5 (br s, 1H), 7.82 (dd, 1H), 7.72-7.63 (m, 2H); MS (ESI) calcd for C₈H₄F₃IO₂: 315.9208. Found: 314.8 (M-H).

### Example 81B

### 2-iodo-3-(trifluoromethyl)benzamide

The title compound was prepared using the procedure described in Example 91A except using 2-iodo-3-(trifluoromethyl)benzoic acid instead of 2-chloro-3-(trifluoromethyl)benzoic acid. ¹H NMR (DMSO-d₆) δ 7.94 (br s, 1H), 7.75 (dd, 1H), 7.68 (br s, 1H), 7.60 (dd, 1H), 7.52 (d, 1H); MS (ESI) calcd for C₈H₅F₃INO: 314.9368. Found: 313.8 (M-H).

### Example 81C

### 2-iodo-3-(trifluoromethyl)benzonitrile

The title compound was prepared using the procedure described in Example 91B except using 2-iodo-3-(trifluoromethyl)benzamide instead of 2-chloro-3-(trifluoromethyl)benzamide. ¹H NMR (CDCl₃) δ 7.86-7.83 (m, 1H), 7.78-7.74 (m, 1H), 7.63-7.57 (m, 1H).

### Example 81D

### 5-[2-iodo-3-(trifluoromethyl)phenyl]-1H-tetraazole

The title compound was prepared using the procedure described in Example 53A except using 2-iodo-3-(trifluoromethyl)benzonitrile instead of 2-fluoro-3-(trifluoromethyl)benzonitrile. ¹H NMR (CDCl₃) δ 7.88-7.82 (m, 2H), 7.64 (dd, 1H); MS (ESI) calcd for C₈H₄F₃IN₄: 339.9433. Found: 339.0 (M-H).

### Example 81E

### 3-({5-[2-iodo-3-(trifluoromethyl)phenyl]-1H-tetraazol-1-yl}methyl)pyridine

The title compound was prepared using the procedure described in Example 9 except using 5-[2-iodo-3-(trifluoromethyl)phenyl]-1H-tetraazole and 3-(bromomethyl)pyridine hydrobromide instead of 5-(2,3-dichlorophenyl)-1H-tetraazole and 4-bromomethylpyridine hydrobromide. MS (ESI) m/z 431.8 (M+H)⁺; ¹H NMR (CDCl₃) δ 8.57 (br s, 1H), 8.25 (br s, 1H), 7.86 (dd, 1H), 7.60-7.49 (m, 2H), 7.28-7.25 (m, 1H), 7.20-7.18 (dd, 1H), 5.44 (s, 2H).

### Example 82

### 3-({5-[3-fluoro-2-(trifluoromethyl)phenyl]-1H-tetraazol-1-yl}methyl)pyridine

### Example 82A

### 3-fluoro-2-(trifluoromethyl)benzamide

The title compound was prepared using the procedure described in Example 91A except using 3-fluoro-2-(trifluoromethyl)benzoic acid instead of 2-chloro-3-(trifluoromethyl)benzoic acid. ¹H NMR (DMSO-d₆) δ 8.05 (br s, 1H), 7.80-7.73 (m, 1H), 7.70 (br s, 1H), 7.56-7.50 (m, 1H), 7.32 (d, 1H); MS (ESI) calcd for C₈H₅F₄NO: 207.0307. Found: 205.9 (M-H).

### Example 82B

### 3-fluoro-2-(trifluoromethyl)benzonitrile

The title compound was prepared using the procedure described in Example 91B except using 3-fluoro-2-(trifluoromethyl)benzamide instead of 2-chloro-3-(trifluoromethyl)benzamide. ¹H NMR (CDCl₃) δ 7.73-7.64 (m, 2H), 7.53-7.46 (m, 1H); MS (ESI) calcd for C₈H₃F₄N: 189.0202. Found: 185.9.

### Example 82C

### 5-[3-fluoro-2-(trifluoromethyl)phenyl]-1H-tetraazole

The title compound was prepared using the procedure described in Example 53A except using 3-fluoro-2-(trifluoromethyl)benzonitrile instead of 2-fluoro-3-(trifluoromethyl)benzonitrile. ¹H NMR (CDCl₃) δ 12.8 (br s, 1H), 7.73-7.67 (m, 1H), 7.53 (d, 1H), 7.44 (dd, 1H); MS (ESI) calcd for C₈H₄F₄N₄: 232.0372.

### Example 82D

### 3-({5-[3-fluoro-2-(trifluoromethyl)phenyl]-1H-tetraazol-1-yl}methyl)pyridine

The title compound was prepared using the procedure described in Example 9 except using 5-[3-fluoro-2-(trifluoromethyl)phenyl]-1H-tetraazole and 3-(bromomethyl)pyridine hydrobromide instead of 5-(2,3-dichlorophenyl)-1H-tetraazole and 4-bromomethylpyridine hydrobromide. MS (ESI) m/z 323.9 (M+H)⁺; ¹H NMR (CDCl₃) δ 8.58 (br d, 1H), 8.25 (br s, 1H), 7.69-7.63 (m, 1H0, 7.56-7.46, m 2H), 7.31-7.26 (m, 1H), 6.97 (d, 1H), 5.41 (s, 2H); Anal calcd for C₁₄H₉F₄N₅: C, 52.02%; H, 2.81%; N, 21.67%. Found: C, 51.89%; H, 2.59%; N, 21.57%.

### Example 83

### 3-{[5-(2,3,4-trichlorophenyl)-1H-tetraazol-1-yl]methyl}pyridine

### Example 83A

### 5-(2,3,4-trichlorophenyl)-1H-tetraazole

The title compound was prepared using the procedure described in Example 53A except using 2,3,4-trichlorobenzonitrile, prepared using the procedure described in Bondinell et. al., J. Med. Chem., 23, 5:506-511 (1980), instead of 2-fluoro-3-(trifluoromethyl)benzonitrile.

### Example 83B

### 3-{[5-(2,3,4-trichlorophenyl)-1H-tetraazol-1-yl]methyl}pyridine

The title compound was prepared using the procedure described in Example 9 except using 5-(2,3,4-trichlorophenyl)-1H-tetraazole and 3-(bromomethyl)pyridine hydrobromide instead of 5-(2,3-dichlorophenyl)-1H-tetraazole and 4-bromomethylpyridine hydrobromide. MS (ESI+) m/z 339.8 (M+H)⁺; ¹H NMR (CDCl₃) δ 8.59 (br s, 1H), 8.32 (br s, 1H), 7.54-7.50 (m, 2H), 7.30-7.26 (m, 1H), 7.10 (d, 1H), 5.48 (s, 2H); Anal calcd for C₁₃H₈Cl₃N₅: C, 45.84%; H, 2.37%; N, 20.56%. Found: C, 45.83%; H, 2.20%; N, 20.57%.

### Example 84

### 3-{[5-(2,3-dichloro-4-fluorophenyl)-1H-tetraazol-1-yl]methyl}pyridine

### Example 84A

### 2,3-dichloro-4-fluorobenzoic acid

The title compound was prepared using the procedure described in Bennetau, Mortier, Moyroud, Guesnet J., Chem. Soc., Perkin Trans. I, 1265 (1995).

### Example 84B

### 2,3-dichloro-4-fluoro-N-(pyridin-3-ylmethyl)benzamide

The title compound was prepared using the procedure described in Example 85B except using 2,3-dichloro-4-fluorobenzoic acid instead of 3-bromo-2-chlorobenzoic acid. ¹H NMR (DMSO-d₆) δ 9.12 (t, 1H), 8.58 (d, 1H), 8.48 (dd, 1H), 7.76 (ddd, 1H), 7.76-7.52 (m, 2H), 7.39 (dd, 1H), 4.48 (d, 2H); MS (ESI) calcd for C₁₃H₉Cl₂FNO: 298.0076. Found: 298.9 (M+H).

### Example 84C

### 3-{[5-(2,3-dichloro-4-fluorophenyl)-1H-tetraazol-1-yl]methyl}pyridine

The title compound was prepared using the procedure described in Example 85C except using 2,3-dichloro-4-fluoro-N-(pyridin-3-ylmethyl)benzamide instead of 3-bromo-2-chloro-N-(pyridin-3-ylmethyl)benzamide. MS (ESI−) m/z 357.9 (M-H)⁻; ¹H NMR (DMSO-d₆) δ 8.66 (dd, 1H), 8.54 (d, 1H), 8.87 (br d, 1H), 7.81-7.70 (m, 2H), 77.60 (dd, 1H), 5.71 (s, 2H); Anal calcd for C₁₃H₉C₁₃FN₅: C, 43.30%; H, 2.52%; N, 19.42%. Found: C, 42.66%; H, 2.22%; N, 19.06%.

### Example 85

### 3-{[5-(3-bromo-2-chlorophenyl)-1H-tetraazol-1-yl]methyl}pyridine

### Example 85A

### 3-bromo-2-chlorobenzoic acid

Concentrated sulfuric acid (7 mL) was treated with sodium nitrite (690 mg, 10.0 mmol) in portions and stirred until the solids dissolved. The mixture was cooled to 0° C. and treated with a solution of 3-amino-2-chloro-benzoic acid (1.54 g, 9.00 mmol) in glacial acetic acid (18 mL) dropwise over 15 minutes. After stirring the mixture at 0° C. for 30 minutes, the mixture was treated dropwise with a solution of 48% aqueous hydrobromic acid (1.5 mL) and copper(I) bromide (2.87 g, 20.0 mmol) precooled to 0° C. After stirring at 0° C. for 30 minutes, the mixture was treated with water (50 mL), filtered, and the filter cake washed with hexanes. The obtained solid was recrystallized from hot water to provide the title compound. ¹H NMR (DMSO-d₆) δ 13.5 (br s, 1H), 7.92 (dd, 1H), 7.72 (dd, 1H), 7.37 (dd, 1H); MS (ESI) calcd for C₇H₄BrClO₂: 233.9083. Found: 235.2 (M+H).

### Example 85B

### 3-bromo-2-chloro-N-(pyridin-3-ylmethyl)benzamide

3-bromo-2-chlorobenzoic acid (589 mg, 2.50 mmol) in anhydrous toluene (7.5 mL) and anhydrous N,N-dimethylformamide (0.4 mL) was treated with thionyl chloride (357 mg, 3.00 mmol) dropwwise. The mixture was stirred for 2 hours at reflux, cooled to room temperature, and concentrated under reduced pressure. The residue was treated with anhydrous toluene (10 mL) followed by successive addition of triethylamine (759 mg, 7.50 mmol) and 3-(aminomethyl)pyridine (406 mg, 3.75 mmol) dropwise. After stirring at room temperature for 1 hour, the mixture was treated with water (10 mL) and extracted with dichloromethane (3×10 mL). The organic extracts were combined, dried over magnesium sulfate, filtered, and the filtrate was concentrated under reduced pressure. The residue was purified by flash chromatography (silica gel, 100% ethyl acetate, product R_{f}˜0.3) to provide the title compound. ¹H NMR (DMSO-d₆) δ 9.11 (t, 1H), 8.57 (d, 1H), 8.48 (dd, 1H), 7.84 (dd, 1H), 7.76 (ddd, 1H), 7.47 (dd, 1H), 7.41-7.32 (m, 2H); MS (ESI) calcd for C₁₃H₁₀BrClN₂O: 323.9665. Found: 324.9 (M+H).

### Example 85C

### 3-{[5-(3-bromo-2-chlorophenyl)-1H-tetraazol-1-yl]methyl}pyridine

3-Bromo-2-chloro-N-(pyridin-3-ylmethyl)benzamide (325 mg, 1.00 mmol) and triphenylphosphine (525 mg, 2.00 mmol) were combined in anhydrous tetrahydrofuran (5 mL) and treated with diisopropylazodicarboxylate (387 μL, 2.00 mmol) followed by azidotrimethylsilane (265 μL, 2.00 mmol). The reaction mixture was stirred at room temperature for 24 hours, diluted with water (10 mL) and extracted with CH₂Cl₂ (3×). The organic extracts were combined, dried (magnesium sulfate), filtered, and the filtrate was concentrated under reduced pressure. The residue was purified by flash chromatography (40% isopropyl alcohol, 60% hexanes) to provide the title compound. MS (ESI+) m/z 351.9 (M+H)⁺; ¹H NMR (CDCl₃) δ 8.56 (br s, 1H), 8.28 (br s, 1H), 7.88 (dd, 1H), 7.51 (br d, 1H), 7.28-7.24 (m, 2H), 7.19 (dd, 1H), 5.48 (s, 2H); Anal calcd for C₁₃H₉BrClN₅: C, 44.53%; H, 2.59%; N, 19.98%. Found: C, 44.78%; H, 2.37%; N, 20.03%.

### Example 86

### 3-{[5-(2-chloro-3-iodophenyl)-1H-tetraazol-1-yl]methyl}pyridine

### Example 86A

### 2-chloro-3-iodobenzoic acid

Concentrated sulfuric acid (6.5 mL) was treated with sodium nitrite (621 mg, 9.00 mmol) in portions and stirred until the solids dissolved. The mixture was cooled to 0° C. and treated with a solution of 3-amino-2-chloro-benzoic acid (1.37 g, 8.00 mmol) in glacial acetic acid (16 mL) dropwise. After stirring at 0° C. for 30 minutes, the mixture was treated with a precooled 0° C. solution of potassium iodide (3.32 g, 20.0 mmol) in water (10 mL) dropwise. After stirring at 0° C. for 2 hours, the mixture was filtered and the filter cake washed with hexanes. The obtained solid was recrystallized from hot water to provide the title compound. ¹H NMR (DMSO-d₆) δ 13.6 (br s, 1H), 8.11-8.08 (m, 1H), 7.70-7.68 (m, 1H), 7.71 (dd, 1H); MS (ESI) calcd for C₇H₄ClO₂: 281.8945. Found: 280.9 (M-H).

### Example 86B

### 2-chloro-3-iodo-N-(pyridin-3-ylmethyl)benzamide

The title compound was prepared using the procedure described in Example 85B except using 2-chloro-3-iodobenzoic acid instead of 3-bromo-2-chlorobenzoic acid. ¹H NMR (DMSO-d₆) δ 9.07 (t, 1H), 8.56 (d, 1H), 8.48 (dd, 1H), 8.02 (dd, 1H), 7.75 (ddd, 1H), 7.45 (dd, 1H), 7.38 (ddd, 1H), 7.15 (dd, 1H), 4.46 (d, 1H); MS (ESI) calcd for C₁₃H₁₀ClIN₂O: 371.9526. Found: 372.9 (M+H).

### Example 86C

### 3-{[5-(2-chloro-3-iodophenyl)-1H-tetraazol-1-yl]methyl}pyridine

The title compound was prepared using the procedure described in Example 85C except using 2-chloro-3-iodo-N-(pyridin-3-ylmethyl)benzamide instead of 3-bromo-2-chloro-N-(pyridin-3-ylmethyl)benzamide. MS (ESI+) m/z 397.9 (M+H)⁺; ¹H NMR (CDCl₃) δ 8.56 (br s, 1H), 8.27 (br s, 1H), 8.11 (dd, 1H), 7.50 (br d, 1H), 7.28-7.22 (m, 1H), 7.21 (dd, 1H), 7.08 (dd, 1H), 5.47 (s, 2H); Anal calcd for C₁₃H₉ClIN₅: C, 39.27%; H, 2.28%; N, 17.61%. Found: C, 39.43%; H, 1.99%; N, 17.55%.

### Example 87

### 3-{[1-(2,3-dichlorophenyl)-1H-tetraazol-5-yl]methyl}pyridine

### Example 87A

### N-(2,3-dichlorophenyl)-2-pyridin-3-ylacetamide

2,3-Dichloroaniline (1.05 g, 6.49 mmol) in anhydrous tetrahydrofuran (30 mL) was treated with triethylamine (1.32 g, 1.81 mL, 13.0 mmol) followed by 3-pyridylacetic acid hydrochloride (1.02 g, 5.90 mmol), followed by hydroxybenzotriazole (877 mg, 6.49 mmol) followed by EDC (1.24 g, 6.49 mmol). The reaction mixture was stirred at room temperature overnight and then heated at 50° C. for 1 hour. After cooling to room temperature, the mixture was treated with water (50 mL) and extracted with dichloromethane (3×50 mL). The organic extracts were combined, dried over sodium sulfate, filtered, and the filtrate was concentrated under reduced pressure. The residue was purified by flash chromatography (silica gel/ethyl acetate, product R_{f}=0.4) to provide the title compound. ¹H NMR (DMSO-d₆) δ 9.98 (s, 1H), 8.54 (d, 1H), 8.47 (dd, 1H), 7.76 (ddd, 1H), 7.66 (dd, 1H), 7.47 (dd, 1H), 7.39-7.31(m, 2H), 3.91 (s, 2H); MS (ESI) calcd for C₁₃H₁₀Cl₂N₂O: 280.0170: Found: 280.9 (M+H)⁺.

### Example 87B

### N-(2,3-dichlorophenyl)-2-pyridin-3-ylethanethioamide

N-(2,3-Dichlorophenyl)-2-pyridin-3-ylacetamide (483 mg, 1.72 mmol) and Lawesson's reagent (366 mg, 877 mmol) were combined in anhydrous toluene (4.3 mL) and heated at reflux for 1.5 hours. After cooling to room temperature, the mixture was treated with water (10 mL) and extracted with dichloromethane (3×20 mL). The organic extracts were combined, dried over sodium sulfate, filtered, and the filtrate was concentrated under reduced pressure. The product was purified by flash chromatography (silica gel/ethyl acetate, product R_{f}=0.5) to provide the title compound. ¹H NMR (DMSO-d₆) δ 11.91 (s, 1H), 8.61 (d, 1H), 8.47 (dd, 1H), 7.83 (ddd, 1H), 7.63 (m, 1H), 7.43-7.36(m, 3H), 4.15 (s, 2H); MS (ESI) calcd for C₁₃H₁₀Cl₂N₂S: 295.9942: Found: 296.9 (M+H)⁺.

### Example 87C

### 3-{[1-(2,3-dichlorophenyl)-1H-tetraazol-5-yl]methyl}pyridine

N-(2,3-Dichlorophenyl)-2-pyridin-3-ylethanethioamide (208 mg, 0.700 mmol) in anhydrous tetrahydrofuran (7 mL) at 0° C. was treated with mercury(II) acetate (446 mg, 1.40 mmol) followed by azidotrimethylsilane (806 mg, 921 ml, 7.00 mmol). The mixture was stirred at 0° C. for 1 hour and treated with saturated ammonium chloride solution (10 mL). The mixture was extracted with dichloromethane (3×10 mL). The organic extracts were combined, dried over sodium sulfate, filtered, and the filtrate was concentrated under reduced pressure. The residue was purified by flash chromatography (silica gel/ethyl acetate, product R_{f}=0.4) to provide the title compound. ¹H NMR (DMSO-d₆) δ 8.43 (dd, 1H), 8.27 (d, 1H), 7.99 (dd, 1H), 7.83 (dd, 1H), 7.65 (dd, 1H), 7.53 (ddd, 1H), 7.28 (ddd, 1H), 4.29 (s, 2H); MS (ESI) calcd for C₁₃H₉Cl₂N₅: 305.0325. Found: 305.9 (M+H)⁺.

### Example 88

### 3-({1-[2-chloro-4-fluoro-3-(trifluoromethyl)phenyl]-1H-tetraazol-5-yl}methyl)pyridine

### Example 88A

### 2-chloro-4-fluoro-1-nitro-3-(trifluoromethyl)benzene

Concentrated nitric acid (12.6 mL) and concentrated sulfuric acid (12.6 mL) were combined at 0° C. and treated with neat 1-chloro-3-fluoro-2-(trifluoromethyl)benzene (5.36 g, 26.5 mmol) dropwise with stirring. The mixture was stirred at 0° C. for 10 minutes, allowed to warm to room temperature over 30 minutes, and poured into a beaker containing ice (50 g). The mixture was extracted with dichloromethane (3×30 mL). The organic extracts were combined, dried over sodium sulfate, filtered, and the filtrate was concentrated under reduced pressure. The residue was purified by flash chromatography (silica gel/10% ethyl acetate in hexanes, product R_{f}=0.5) to provide an inseparable mixture of the title compound and a regioisomer. ¹H NMR (CDCl₃) major regioisomer δ 7.97 (dd, 1H), 7.33 (dd, 1H); minor regioisomer δ 8.18 (dd, 1H), 7.53 (d, 1H).

### Example 88B

### 2-chloro-4-fluoro-3-(trifluoromethyl)phenylamine

Absolute ethanol (10 mL) and glacial acetic acid (20 mL) were combined and treated with the mixture of regioisomers (1.46 g, 6.00 mmol) from Example 88A, followed by addition of fine mesh iron powder (1.73 g, 30.0 mmol) portionwise. The mixture was heated at reflux for 30 minutes, cooled to room temperature, filtered, and the filter cake was washed with dichloromethane and methanol. The filtrate was concentrated under reduced pressure, treated with 10% methanol in dichloromethane (25 mL) and washed with saturated sodium bicarbonate solution. The organic layer was dried over sodium sulfate, filtered, and the filtrate was concentrated under reduced pressure. The residue was purified by flash chromatography (silica gel/10% ethyl acetate in hexanes) to provide an inseparable mixture of the title compound and a regioisomer. ¹H NMR (DMSO-d₆) major regioisomer δ 7.18 (dd, 1H), 7.10 (dd, 1H); minor regioisomer δ 7.18 (d, 1H), 7.01 (d, 1H). MS (ESI) calcd for C₇H₄ClF₄N, 212.9968. Found: 211.9 (M-H)⁻.

### Example 88C

N-[2-chloro-4-fluoro-3-(trifluoromethyl)phenyl]-2-pyridin-3-ylacetamide

The title compound was prepared using the procedure described in Example 87A except using 2-chloro-4-fluoro-3-(trifluoromethyl)phenylamine instead of 2,3-dichloroaniline. ¹H NMR (DMSO-d₆) δ 10.10 (s, 1H), 8.54 (d, 1H), 8.47 (dd, 1H), 7.94 (dd, 1H), 7.76 (ddd, 1H), 7.50 (dd, 1H), 7.36 (dd, 1H), 3.80 (s, 2H); MS (ESI) calcd for C₁₄H₉ClF₄N₂O: 332.034. Found: 332.9 (M+H)⁺.

### Example 88D

### N-[2-chloro-4-fluoro-3-(trifluoromethyl)phenyl]-2-pyridin-3-ylethanethioamide

The title compound was prepared using the procedure described in Example 87B except using N-[2-chloro-4-fluoro-3-(trifluoromethyl)phenyl]-2-pyridin-3-ylacetamide instead of N-(2,3-dichlorophenyl)-2-pyridin-3-ylacetamide. ¹H NMR (DMSO-d₆) δ 11.92 (s, 1H), 8.61 (d, 1H), 8.48 (dd, 1H), 7.86-7.81 (m, 2H), 7.58 (dd, 1H), 7.38 (dd, 1H), 4.17 (s, 2H); MS (ESI) calcd for C₁₄H₉ClF₄N₂S: 348.0111. Found: 348.9 (M+H)⁺.

### Example 88E

### 3-({1-[2-chloro-4-fluoro-3-(trifluoromethyl)phenyl]-1H-tetraazol-5-yl}methyl)pyridine

The title compound was prepared using the procedure described in Example 87C except using N-[2-chloro-4-fluoro-3-(trifluoromethyl)phenyl]-2-pyridin-3-ylethanethioamide instead of N-(2,3-dichlorophenyl)-2-pyridin-3-ylethanethioamide. ¹H NMR (CDCl₃) δ 8.48 (dd, 1H), 8.36 (d, 1H), 8.28 (dd, 1H), 7.87 (dd, 1H), 7.66 (d, 1H), 7.38 (dd, 1H), 4.33 (s, 2H); MS (ESI) calcd for C₁₄H₉Cl₂F₄N₅: 393.0171. Found: 357.9 (M-Cl)⁻; Anal calcd for C₁₄H₉Cl₂F₄N₅: C, 46.66% H, 2.30%; N, 17.77%. Found: C, 48.91%; H, 3.01%; N, 17.24%.

### Example 89

### 3-({1-[2-chloro-3-(trifluoromethyl)phenyl]-1H-tetraazol-5-yl}methyl)pyridine

### Example 89A

### tert-butyl 2-chloro-3-(trifluoromethyl)phenylcarbamate

2-Chloro-3-trifluromethylbenzoic acid (2.85 g, 12.7 mmol) and triethylamine (1.41 g, 14 mmol) were combined in tert-butanol and treated with diphenylphosphorylazide (3.85 g, 14 mmol). The reaction mixture was refluxed for 12 hours, cooled, and concentrated under reduced pressure. The residue was treated with ethyl acetate (100 mL) and washed with water, 5% citric acid, water, saturated NaHCO₃, brine, dried (MgSO₄), filtered, and the filtrate was concentrated under reduced pressure. The residue was purified by flash chromatography (5% ethyl acetate/hexane) to provide the title compound.

### Example 89B

### 2-chloro-3-(trifluoromethyl)aniline

tert-Butyl 2-chloro-3-(trifluoromethyl)phenylcarbamate (1 g, 3.38 mmol) in diethyl ether (2 mL) was treated with 2M HCl/diethyl ether (10 mL, 20 mmol), stirred at room temperature for 24 hours, diluted with diethyl ether (50 mL), and filtered to provide the title compound as the hydrochloride salt. ¹H NMR (DMSO-d₆) δ 7.20 (dd, 1H), 7.09 (dd, 1H), 6.96 (dd, 1H), 6.11 (s, 3H).

### Example 89C

### N-[2-chloro-3-(trifluoromethyl)phenyl]-2-pyridin-3-ylacetamide

The title compound was prepared using the procedure described in Example 87A except using 2-chloro-3-(trifluoromethyl)aniline instead of 2,3-dichloroaniline. ¹H NMR (DMSO-d₆) δ 10.09 (s, 1H), 8.55 (d, 1H), 8.47 (dd, 1H), 7.96 (dd, 1H), 7.77 (ddd, 1H), 7.69 (dd, 1H), 7.54 (dd, 1H), 7.37 (ddd, 1H), 3.83 (s, 2H); MS (ESI) calcd for C₁₄H₁₀ClF₃N₂O: 314.0434. Found: 315.0 (M+H)⁺.

### Example 89D

### N-[2-chloro-3-(trifluoromethyl)phenyl]-2-pyridin-3-ylethanethioamide

The title compound was prepared using the procedure described in Example 87B except using N-[2-chloro-3-(trifluoromethyl)phenyl]-2-pyridin-3-ylacetamide instead of N-(2,3-dichlorophenyl)-2-pyridin-3-ylacetamide. ¹H NMR (DMSO-d₆) δ 11.94 (s, 1H), 8.62 (dd, 1H), 7.87-7.84 (m, 2H), 7.77 (d, 1H), 7.62 (dd, 1H), 7.39 (dd, 1H), 4.18 (s, 2H); MS (ESI) calcd for C₁₄H₁₀ClF₃N₂S: 330.0205. Found: 330.9 (M+H)⁺.

### Example 89E

### 3-({1-[2-chloro-3-(trifluoromethyl)phenyl]-1H-tetraazol-5-yl}methyl)pyridine

The title compound was prepared using the procedure described in Example 87C except using N-[2-chloro-3-(trifluoromethyl)phenyl]-2-pyridin-3-ylethanethioamide instead of N-(2,3-dichlorophenyl)-2-pyridin-3-ylethanethioamide. ¹H NMR (CDCl₃) δ 8.63 (dd, 1H), 8.53 (d, 1H), 8.23-8.19 (m, 2H), 7.97 (d, 1H), 7.88 (dd, 1H), 7.72 (dd, 1H), 4.42 (s, 2H); MS (ESI) calcd for C14H10Cl2F3N5: 375.0265. Found: 339.9 (M-35); Anal calcd for C₁₄H₁₀Cl₂F₃N₅: C, 44.70%; H, 2.68%; N, 18.62%. Found: C, 44.64%; H, 2.42%; N, 18.68%.

### Example 90

### 3-{[1-(2,3-dichloro-4-fluorophenyl)-1H-tetraazol-5-yl]methyl}pyridine

### Example 90A

### tert-butyl 2,3-dichloro-4-fluorophenylcarbamate

The title compound was prepared using the procedure described in Example 89A except using 2,3-dichloro-4-fluorobenzoic acid, prepared using the procedure described in Bennetau, Mortier, Moyroud, Guesnet J., Chem. Soc., Perkin Trans. I, 1265 (1995), instead of 2-chloro-3-trifluromethylbenzoic acid. ¹H NMR (DMSO-d₆) δ 7.13 (dd, 1H), 6.77 (dd, 1H), 5.53 (s, 2H); MS (ESI) calcd for C₆H₄Cl₂FN: 178.9705. Found: 177.9 (M-H).

### Example 90B

### 2,3-dichloro-4-fluoroaniline

The title compound was prepared using the procedure described in Example 89B except using tert-butyl 2,3-dichloro-4-fluorophenylcarbamate instead of tert-butyl 2-chloro-3-(trifluoromethyl)phenylcarbamate.

### Example 90C

### N-(2,3-dichloro-4-fluorophenyl)-2-pyridin-3-ylacetamide

The title compound was prepared using the procedure described in Example 87A except using 2,3-dichloro-4-fluoroaniline instead of 2,3-dichloroaniline. ¹H NMR (DMSO-d₆) δ 10.11 (s, 1H), 8.54 (d, 1H), 8.47 (dd, 1H), 7.75 (ddd, 1H), 7.65 (dd, 1H), 7.45 (dd, 1H), 7.37 (ddd, 1H), 3.83 (s, 2H); MS (ESI) calcd for C₁₃H₉Cl₂FN₂O: 298.0076. Found: 298.9 (M+H).

### Example 90D

### N-(2,3-dichloro-4-fluorophenyl)-2-pyridin-3-ylethanethioamide

The title compound was prepared using the procedure described in Example 87B except using N-(2,3-dichloro-4-fluorophenyl)-2-pyridin-3-ylacetamide instead of N-(2,3-dichlorophenyl)-2-pyridin-3-ylacetamide. ¹H NMR (DMSO-d₆) δ 11.91 (s, 1H), 8.60 (d, 1H), 8.48 (dd, 1H), 7.83 (ddd, 1H), 7.56-7.46 (m, 2H), 7.38 (ddd, 1H), 4.15 (s, 2H).

### Example 90E

### 3-{[1-(2,3-dichloro-4-fluorophenyl)-1H-tetraazol-5-yl]methyl}pyridine

The title compound was prepared using the procedure described in Example 87C except using N-(2,3-dichloro-4-fluorophenyl)-2-pyridin-3-ylethanethioamide instead of N-(2,3-dichlorophenyl)-2-pyridin-3-ylethanethioamide. ¹H NMR (CDCl₃) δ 8.67 (dd, 1H), 8.59 (d, 1H), 8.04 (d, 1H), 7.97 (dd, 1H), 7.82 (dd, 1H), 7.68 (dd, 1H), 4.40 (s, 2H); MS (ESI) calcd for C₁₃H₉Cl₃FN₅: 358.9908. Found: 323.9 (M-35). Anal calcd for C₁₃H₉Cl₃FN₅: C, 43.30%; H, 2.52%; N, 19.42%. Found: C, 43.07%; H, 2.31%; N, 19.40%.

### Example 91

### 3-({5-[2-chloro-3-(trifluoromethyl)phenyl]-1H-tetraazol-1-yl}methyl)pyridine

### Example 91A

### 2-chloro-3-(trifluoromethyl)benzamide

2-Chloro-3-(trifluoromethyl)benzoic acid (4.49 g, 20.0 mmol) in anhydrous toluene (25 mL) and anhydrous N,N-dimethylformamide (0.5 mL) was treated with thionyl chloride (2.19 mL, 30.0 mmol) dropwise and then heated at reflux with stirring for 2 hours. After cooling to room temperature, the volatiles were removed under reduced pressure and the residue was treated with a cold solution of aqueous ammonium hydroxide (25 mL) dropwise. The mixture was allowed to warm to room temperature, filtered, and the filter cake was dried under reduced pressure to provide the title compound. ¹H NMR (CDCl₃) δ 7.80 (d, 2H), 7.46 (dd, 1H), 6.24 (br s, 1H), 6.12 (br s, 1H); MS (ESI) calcd for C₈H₅ClF₃NO: 223.0012. Found: 221.9 (M-H).

### Example 91B

### 2-chloro-3-(trifluoromethyl)benzonitrile

2-Chloro-3-(trifluoromethyl)benzamide (2.24 g, 10.0 mmol) in toluene (20 mL) and thionyl chloride (1.46 mL, 20 mmol) was heated at reflux with stirring overnight. The mixture was cooled in an ice bath, treated with water (20 mL) dropwise, and extracted with dichloromethane (3×20 mL). The organic extracts were combined, dried over magnesium sulfate, filtered, and the filtrate was concentrated under reduced pressure. The residue was purified by flash chromatography (silica gel, 20% ethyl acetate in hexanes, product Rf˜0.3) to provide the title compound. ¹H NMR (CDCl₃) δ 7.97-7.86 (m, 2H), 7.53 (ddq, 1H).

### Example 91C

### 5-[2-chloro-3-(trifluoromethyl)phenyl]-1H-tetraazole

The title compound was prepared using the procedure described in Example 53A except using 2-chloro-3-(trifluoromethyl)benzonitrile instead of 2-fluoro-3-(trifluoromethyl)benzonitrile. ¹H NMR (CDCl₃) δ 12.9 (br s, 1H), 8.49 (d, 1H), 7.95 (d, 1H), 7.63 (dd, 1H); MS (ESI) calcd for C₈H₄ClF₃N₄: 248.0077. Found: 248.8 (M+H).

### Example 91D

### 3-({5-[2-chloro-3-(trifluoromethyl)phenyl]-1H-tetraazol-1-yl}methyl)pyridine

The title compound was prepared using the procedure described in Example 9 except using 5-[2-chloro-3-(trifluoromethyl)phenyl]-1H-tetraazole and 3-bromomethylpyridine hydrobromide instead of 5-(2,3-dichlorophenyl)-1H-tetraazole and 4-bromomethylpyridine hydrobromide. ¹H NMR (CDCl₃) δ 8.55 (br s, 1H), 8.25 (br s, 1H), 7.96 (dd, 1H), 7.54-7.47 (m, 2H), 7.40 (dd, 1H), 7.26-7.22 (m, 1H), 5.50 (s, 2H); MS (ESI) calcd for C₁₄H₉ClF₃N₅: 339.0499. Found: 339.9 (M+H).

Biological Activity

In Vitro Data—P2X₇ Inducedp Secretion of IL-1β

Activation of P2X₇ receptors also induces secretion of IL-1β (Verhoef et al., above; Brough et al., Molecular and Cellular Neuroscience Vol. 19, pages 272-280, 2002). THP-1 cells were plated in 24-well plates at a density of 1×10⁶ cells/well/ml. On the day of the experiment, cells were differentiated with 25 ng/ml LPS and 10 ng/ml final concentration of γIFN for 3 hours at 37° C. In the presence of the differentiation media, the cells were incubated with the antagonists of the present invention for 30 minutes at 37° C. followed by a challenge with 1 mM BzATP for an additional 30 minutes at 37° C. Supernatants of the samples were collected after a 5 minutes centrifugation in microfuge tubes to pellet the cells and debris and to test for mature IL-1β released into the supernatant using either R & D Systems Human IL-1β ELISA assay or Endogen Human IL-1β ELISA, following the manufacturer's instructions. The concentration of the antagonists that inhibited 50% of the agonist-release of IL-1β was expressed as IC₅₀. Representative compounds of the present invention exhibited IC₅₀ values from 0.207 to 2.461 μM.

In Vivo Data—Determination of Antinocicentive Effect

Adult male Sprague-Dawley rats (250-300 g), Charles River Laboratories, Portage, Mich. were used in this study. Animal handling and experimental protocols were approved by the Institutional Animal Care and Use Committee (IACUC) at Abbott Laboratories. For all surgical procedures, animals were maintained under halothane anesthesia (4% to induce, 2% to maintain), and the incision sites were sterilized using a 10% povidone-iodine solution prior to and after surgeries.

A model of spinal nerve ligation-induced neuropathic pain was produced using the procedure originally described by Kim and Chung (Kim and Chung 1992). The left L5 and L6 spinal nerves of the rat were isolated adjacent to the vertebral column and tightly ligated with a 5-0 silk suture distal to the DRG, and care was taken to avoid injury of the L4 spinal nerve. Sham rats underwent the same procedure, but without nerve ligation. All animals were allowed to recover for at least 1 week and not more than 3 weeks prior to assessment of mechanical allodynia.

Mechanical allodynia was measured using calibrated von Frey filaments (Stoelting, Wood Dale, Ill.). Rats were placed into inverted individual plastic containers (20×12.5×20 cm) on top of a suspended wire mesh grid, and acclimated to the test chambers for 20 min. The von Frey filaments were presented perpendicularly to the plantar surface of the selected hind paw, and then held in this position for approximately 8 sec with enough force to cause a slight bend in the filament. Positive responses included an abrupt withdrawal of the hind paw from the stimulus, or flinching behavior immediately following removal of the stimulus. A 50% withdrawal threshold was determined using an up-down procedure (Dixon 1980). Prior to compound administration, animals demonstrating motor deficit or failure to exhibit subsequent mechanical allodynia were excluded from further studies.

Thirty minutes after intraperitoneal injection in appropriate vehicle, the effects of P2X7 receptor antagonists on mechanical allodynia observed after spinal nerve injury were evaluated. Representative compounds of the present invention exhibited ED₅₀ values between 44 and 232 μmol/kg, obtained from dose-response curves and intraperitoneal administration.

What is claimed is:

1. A method of treating neuropathic pain, chronic inflammatory pain, inflammation, neurodegeneration and promoting neuroregeneration comprising administering a therapeutically effective amount of a compound that is a selective P2X₇ receptor antagonist of formula I 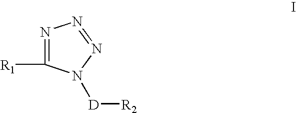
or a pharmaceutically acceptable salt, prodrug, or salt of a prodrug thereof, wherein
D is a bond or C₁₋₅ alkylene;
R₁ is selected form the groups consisting of cycloalkyl, cycloalkenyl, heterocycle, aryl, heteroaryl, arylalkyl and heteroarylalkyl; wherein each R₁ is substituted with 0, 1, 2, 3, 4 or 5 substituents independently selected from the group consisting of alkyl, alkenyl, alkynyl, nitro, cyano, halo, —OR_{c}, —O(CO)R_{c}, —OC(O)OR_{c}, —OS(O)₂R_{c}, —SR_{c}, —S(O)R_{c}, —S(O)₂R_{c}, —S(O)₂OR_{c}, —S(O)₂NR_{c}R_{d}, —NR_{c}R_{d}, —N(R_{d})C(O)OR_{c}, —N(R_{d})C(O)NR_{c}R_{d}, —N(R_{d})S(O)₂NR_{c}R_{d}, —C(O)R_{c}, —C(O)OR_{c}, —C(O)NR_{c}R_{d}, haloalkyl, cyanoalkyl, nitroalkyl, -alkylOR_{c}, —O(CO)R_{c}, -alkylOC(O)OR_{c}, -alkylOS(O)₂R_{c}, -alkylSR_{c}, -alkylS(O)R_{c}, -alkylS(O)₂R_{c}, -alkylS(O)₂OR_{c}, -alkylS(O)₂NR_{c}R_{d}, -alkylNR_{c}R_{d}, -alkylN(R_{d})C(O)OR_{c}, -alkylN(R_{d})C(O)NR_{c}R_{d}, -alkylN(R_{d})S(O)₂NR_{c}R_{d}, -alkylC(O)R_{c}, -alkylC(O)OR_{c}, -alkylC(O)NR_{c}R_{d} and R₃; provided that when R₁ is arylalkyl or heteroarylalkyl, D is a bond;
R₂ is selected form the groups consisting of cycloalkyl, cycloalkenyl, heterocycle, aryl, and heteroaryl; wherein each R₂ is substituted with 0, 1, 2, 3, 4 or 5 substituents independently selected from the group consisting of alkyl, alkenyl, alkynyl, nitro, cyano, halo, —OR_{c}, —O(CO)R_{c}, —OC(O)OR_{c}, —OS(O)₂R_{c}, —SR_{c}, —S(O)R_{c}, —S(O)₂R_{c}, —S(O)₂OR_{c}, —S(O)₂NR_{c}R_{d}, —NR_{c}R_{d}, —N(R_{d})C(O)OR_{c}, —N(R_{d})C(O)NR_{c}R_{d}, —N(R_{d})S(O)₂NR_{c}R_{d}, —C(O)R_{c}, —C(O)OR_{c}, —C(O)NR_{c}R_{d}, haloalkyl, cyanoalkyl, nitroalkyl, -alkylOR_{c}, —O(CO)R_{c}, -alkylOC(O)OR_{c}, -alkylOS(O)₂R_{c}, -alkylSR_{c}, -alkylS(O)R_{c}, -alkylS(O)₂R_{c}, -alkylS(O)₂OR_{c}, -alkylS(O)₂NR_{c}R_{d}, -alkylNR_{c}R_{d}, -alkylN(R_{d})C(O)OR_{c}, -alkylN(R_{d})C(O)NR_{c}R_{d}, -alkylN(R_{d})S(O)₂NR_{c}R_{d}, -alkylC(O)R_{c}, -alkylC(O)OR_{c}, -alkylC(O)NR_{c}R_{d} and R₃;
R₃ is selected from the group consisting of cycloalkyl, cycloalkenyl, heterocycle, aryl, and heteroaryl; wherein each R₃ is independently substituted with 0, 1, 2, 3, 4 or 5 substituents independently selected from the group consisting of alkyl, alkenyl, alkynyl, nitro, cyano, halo, formyl, hydroxy, alkoxy, haloalkoxy, —OC(O)alkyl, —S(O)₂alkyl, —S(O)₂NH₂, —S(O)₂N(H)(alkyl), —S(O)₂N(alkyl)₂, —NH₂, —N(H)(alkyl), —N(alkyl)₂, —C(O)(alkyl), —C(O)(OH), —C(O)(Oalkyl), —C(O)NH₂, —C(O)N(H)(alkyl), —C(O)N(alkyl)₂, haloalkyl, formylalkyl, cyanoalkyl, nitroalkyl, hydroxyalkyl, alkoxyalkyl, haloalkoxyalkyl, -alkylOC(O)alkyl, -alkyl-S(O)₂alkyl, -alkyl-S(O)₂NH₂, -alkyl-S(O)₂N(H)(alkyl), -alkyl-S(O)₂N(alkyl)₂, -alkyl-NH₂, -alkyl-N(H)(alkyl), -alkyl-N(alkyl)₂, -alkyl-C(O)(alkyl), -alkyl-C(O)(OH), -alkyl-C(O)(Oalkyl), -alkyl-C(O)NH₂, -alkyl-C(O)N(H)(alkyl), and -alkyl-C(O)N(alkyl)₂; and
R_{c} and R_{d}, at each occurrence, are independently selected from the group consisting of hydrogen, alkyl, haloalkyl, aryl, heteroaryl, arylalkyl and heteroarylalkyl; wherein the aryl, heteroaryl, aryl moiety of the arylalkyl and heteroaryl moiety of the heteroarylalkyl are independently substituted with 0, 1, 2 or 3 substituents independently selected from the group consisting of alkyl, halo, haloalkyl, hydroxyl, hydroxyalkyl and alkoxyalky.

2. The method according to claim 1, wherein
R₁ is aryl;
R₂ is aryl, and
D is a C₁₋₅ alkylene.

3. The method according to claim 2, wherein
R₁ is phenyl.

4. The method according to claim 3, wherein
R₂ is phenyl.

5. The method of claim 4, wherein the compound is selected from the group consisting of
1-(2,6-dichlorobenzyl)-5-(2,3-dichlorophenyl)-1H-tetraazole,
1-benzyl-5-(2,3-dichlorophenyl)-1H-tetraazole,
1-(4-chlorobenzyl)-5-(2,3-dichlorophenyl)-1H-tetraazole,
1-(2,4-dichlorobenzyl)-5-(2,3-dichlorophenyl)-1H-tetraazole,
1-(2-chlorobenzyl)-5-(2,3-dichlorophenyl)-1H-tetraazole,
5-(2,3-dichlorophenyl)-1-(2-methoxybenzyl)-1H-tetraazole,
5-(2,3-dichlorophenyl)-1-(2-methoxybenzyl)-1H-tetraazole,
methyl 4-{[5-(2,3-dichlorophenyl)-1H-tetraazol-1-yl]methyl}benzoate,
5-(2,3-dichlorophenyl)-1-(2-fluorobenzyl)-1H-tetraazole,
5-(2,3-dichlorophenyl)-1-(2-bromobenzyl)-1H-tetraazole, 5-(2,3-dichlorophenyl)-1-(2-methylbenzyl)-1H-tetraazole,
1-(1,1′-biphenyl-2-ylmethyl)-5-(2,3-dichlorophenyl)-1H-tetraazole, 5-(2,3-dichlorophenyl)-1-(2-iodobenzyl)-1H-tetraazole,
1-benzyl-5-(2-chlorophenyl)-1H-tetraazole,
1-(2-chlorobenzyl)-5-(2-chlorophenyl)-1H-tetraazole,
5-(2-chlorophenyl)-1-(2,6-dichlorobenzyl)-1H-tetraazole,
1-(4-chlorobenzyl)-5-(2-chlorophenyl)-1H-tetraazole,
5-(2-chlorophenyl)-1-(2,4-dichlorobenzyl)-1H-tetraazole,
1-(2-chlorobenzyl)-5-(2,3-dimethoxyphenyl)-1H-tetraazole,
1-(2-chlorobenzyl)-5-(2,3-dimethoxyphenyl)-1H-tetraazole,
2-{[5-(2,3-dichlorophenyl)-1H-tetraazol-1-yl]methyl}phenyl acetate,
5-(2,3-dichlorophenyl)-1-(2-phenylethyl)-1H-tetraazole,
5-(2,3-dichlorophenyl)-1-(3-phenylpropyl)-1H-tetraazole,
5-[2-fluoro-3-(trifluoromethyl)phenyl]-1-(2-methylbenzyl)-1H-tetraazole,
1-(2-methylbenzyl)-5-(2-methylphenyl)-1H-tetraazole,
5-(2,3-dimethylphenyl)-1-(2-methylbenzyl)-1H-tetraazole,
5-(2,3-difluorophenyl)-1-(2-methylbenzyl)-1H-tetraazole,
1-benzyl-5-(2,6-dichlorophenyl)-1H-tetraazole,
5-(2,6-dichlorophenyl)-1-[2-(trifluoromethyl)benzyl]-1H-tetraazole,
1-benzyl-5-(2,3-difluorophenyl)-1H-tetraazole,
5-(2,3-difluorophenyl)-1-[2-(trifluoromethyl)benzyl]-1H-tetraazole,
1-benzyl-5-(2,3-dimethylphenyl)-1H-tetraazole,
5-(2,3-dimethylphenyl)-1-[2-(trifluoromethyl)benzyl]-1H-tetraazole,
5-(2,3-difluorophenyl)-1-(2-methylbenzyl)-1H-tetraazole,
5-(3-chloro-2-fluorophenyl)-1-(2-methylbenzyl)-1H-tetraazole,
1-(2-methylbenzyl)-5-[2-(trifluoromethyl)phenyl]-1H-tetraazole,
1-benzyl-5-[2-(trifluoromethyl)phenyl]-1H-tetraazole,
1-benzyl-5-(2-bromophenyl)-1H-tetraazole,
1-benzyl-5-(3-bromophenyl)-1H-tetraazole,
1-benzyl-5-(4-bromo-2,3,5,6-tetrafluorophenyl)-1H-tetraazole,
1-benzyl-5-(3-iodophenyl)-1H-tetraazole, and
1-benzyl-5-(3-bromo-4-fluorophenyl)-1H-tetraazole.

6. The method according to claim 1, wherein
R₁ is aryl,
R₂ is heteroaryl, and
D is a C₁₋₅ alkylene.

7. The method of claim 6, wherein R₂ is selected from the group consisting of pyridinyl, thiazolyl, imidazolyl, isoxazolyl, thienyl and quinolinyl.

8. The method of claim 7, wherein
R₂ is pyridinyl, and
R₁ is phenyl.

9. The method of claim 8, wherein the compound is selected from the group consisting of
2-{[5-(2,3-dichlorophenyl)-1H-tetraazol-1-yl]methyl}pyridine,
3-{[5-(2,3-dichlorophenyl)-1H-tetraazol-1-yl]methyl}pyridine,
4-{[5-(2,3-dichlorophenyl)-1H-tetraazol-1-yl]methyl}pyridine,
3-{[5-(2-chlorophenyl)-1H-tetraazol-1-yl]methyl}pyridine,
4-{[5-(2-chlorophenyl)-1H-tetraazol-1-yl]methyl}pyridine,
3-{[5-(2,3-dichlorophenyl)-1H-tetraazol-1-yl]methyl}-2-methylpyridine hydrochloride,
3-{[5-(2,3-dichlorophenyl)-1H-tetraazol-1-yl]methyl}-4-methylpyridine,
3-{[5-(2,3-dichlorophenyl)-1H-tetraazol-1-yl]methyl}-2,4-dimethylpyridine,
3-{[5-(2,5-dichlorophenyl)-1H-tetraazol-1-yl]methyl}pyridine hydrochloride,
3-{[5-(2,5-dichlorophenyl)-1H-tetraazol-1-yl]methyl}-4-methylpyridine,
3-{[5-(3,4-dichlorophenyl)-1H-tetraazol-1-yl]methyl}pyridine,
3-{[5-(3,4-dichlorophenyl)-1H-tetraazol-1-yl]methyl}-4-methylpyridine,
3-{[5-(3,4-dichlorophenyl)-1H-tetraazol-1-yl]methyl}-2-methylpyridine,
3-{[5-(2,5-dichlorophenyl)-1H-tetraazol-1-yl]methyl}-2-methylpyridine,
3-{2-[5-(2,3-dichlorophenyl)-1H-tetraazol-1-yl]ethyl}pyridine,
3-{[5-(2,3-difluorophenyl)-1H-tetraazol-1-yl]methyl}pyridine,
3-({5-[2-fluoro-3-(trifluoromethyl)phenyl]-1H-tetraazol-1-yl}methyl)pyridine,
3-({5-[2-fluoro-3-(trifluoromethyl)phenyl]-1H-tetraazol-1-yl}methyl)-2-methylpyridine,
3-({5-[2-(trifluoromethyl)phenyl]-1H-tetraazol-1-yl}methyl)pyridine,
3-({5-[3-(trifluoromethyl)phenyl]-1H-tetraazol-1-yl}methyl)pyridine,
3-{[5-(3-chloro-2,4-difluorophenyl)-1H-tetraazol-1-yl]methyl}pyridine,
3-({5-[2-iodo-3-(trifluoromethyl)phenyl]-1H-tetraazol-1-yl}methyl)pyridine,
3-({5-[3-fluoro-2-(trifluoromethyl)phenyl]-1H-tetraazol-1-yl}methyl)pyridine,
3-{[5-(2,3,4-trichlorophenyl)-1H-tetraazol-1-yl]methyl}pyridine,
3-{[5-(2,3-dichloro-4-fluorophenyl)-1H-tetraazol-1-yl]methyl}pyridine,
3-{[5-(3-bromo-2-chlorophenyl)-1H-tetraazol-1-yl]methyl}pyridine,
3-{[5-(2-chloro-3-iodophenyl)-1H-tetraazol-1-yl]methyl}pyridine, and
3-({5-[2-chloro-3-(trifluoromethyl)phenyl]-1H-tetraazol-1-yl}methyl)pyridine.

10. The method of claim 7, wherein
R₁ is phenyl and R₂ is thiazolyl.

11. The method of claim 10, wherein the compound is selected from the group consisting of
5-(2,3-dichlorophenyl)-1-(1,3-thiazol-4-ylmethyl)-1H-tetraazole,
5-(2,3-dichlorophenyl)-1-[(2-methyl-1,3-thiazol-4-yl)methyl]-1H-tetraazole, and
5-(2,3-dichlorophenyl)-1-[(2,4-dimethyl-1,3-thiazol-5-yl)methyl]-1H-tetraazole.

12. The method of claim 7, wherein
R₁ is phenyl and R₂ is imidazolyl.

13. The method of claim 12, wherein the compound is selected from the group consisting of
5-(2,3-dichlorophenyl)-1-(1H-imidazol-4-ylmethyl)-1H-tetraazole,
5-(2,3-dichlorophenyl)-1-[(5-methyl-1H-imidazol-4-yl)methyl]-1H-tetraazole, and
5-(2,3-dichlorophenyl)-1-[(1-methyl-1H-imidazol-5-yl)methyl]-1H-tetraazole.

14. The method of claim 7, wherein
R₁ is phenyl and R₂ is isoxazolyl.

15. The method of claim 14, wherein the compound is selected from the group consisting of
ethyl 4-{[5-(2,3-dichlorophenyl)-1H-tetraazol-1-yl]methyl}isoxazole-3-carboxylate, and
5-(2,3-dichlorophenyl)-1-[(3,5-dimethylisoxazol-4-yl)methyl]-1H-tetraazole.

16. The method of claim 7, wherein
R₁ is phenyl and R₂ is thienyl.

17. The method of claim 16, wherein the compound is selected from the group consisting of
5-(2,3-dichlorophenyl)-1-(thien-3-ylmethyl)-1H-tetraazole, and
N-{[2-({5-[2-chloro-3-(trifluoromethyl)phenyl]-1H-tetraazol-1-yl}methyl)thien-3-yl]methyl}-N,N-dimethylamine.

18. The method of claim 7, wherein
R₁ is phenyl and R₂ is quinolinyl.

19. The method of claim 18, wherein the compound is
3-({5-[2-chloro-3-(trifluoromethyl)phenyl]-1H-tetraazol-1-yl}methyl)quinoline.

20. The method according to claim 1, wherein
R₁ is heteroaryl,
R₂ is aryl, and
D is C₁₋₅ alkylene.

21. The method according to claim 20, wherein
R₁ is selected from the group consisting of pyridinyl and thienyl, and
R₂ is phenyl.

22. The method of claim 21, wherein
R₁ is pyridinyl.

23. The compound of claim 22, wherein the compound is
3-[1-(2-chlorobenzyl)-1H-tetraazol-5-yl]pyridine.

24. The method according to claim 21, wherein
R₁ is thienyl

25. The method of claim 24, wherein the compound is selected from the group consisting of
1-benzyl-5-(5-chlorothien-2-yl)-1H-tetraazole, and
1-benzyl-5-(5-bromothien-2-yl)-1H-tetraazole.

26. The method according to claim 1, wherein
R₁ and R₂ are independently selected form the groups consisting of aryl and heteroaryl, and
D is a bond.

27. The method of claim 26, wherein the compound is
3-[5-(2,3-dichlorophenyl)-1H-tetraazol-1-yl]pyridine.

28. The method according to claim 1, wherein
R₁ is heteroarylalkyl,
R₂ is aryl, and
D is a bond.

29. The method of claim 28, wherein the compound is
3-{[1-(2,3-dichlorophenyl)-1H-tetraazol-5-yl]methyl}pyridine,
3-({1-[2-chloro-4-fluoro-3-(trifluoromethyl)phenyl]-1H-tetraazol-5-yl}methyl)pyridine,
3-({1-[2-chloro-3-(trifluoromethyl)phenyl]-1H-tetraazol-5-yl}methyl)pyridine, and
3-{[1-(2,3-dichloro-4-fluorophenyl)-1H-tetraazol-5-yl]methyl}pyridine.

30. A method of treating a condition involving the P2X₇ receptor, selected from the group consisting of neuropathic pain, chronic inflammatory pain, neurodegeneration, and inflammation, in a host mammal in need of such treatment comprising administering a therapeutically effective amount of a compound of formula I as defined in claim 1, or a pharmaceutically acceptable salt or prodrug thereof, in combination with a pharmaceutically acceptable carrier.

31. The method according to claim 30, wherein the condition is neuropathic pain.

32. The method according to claim 30, wherein the condition is chronic inflammatory pain.

33. The method according to claim 30, wherein the condition is neurodegeneration.

34. The method according to claim 30, wherein the condition is inflammation.

35. A method of promoting neuroregeneration in a host mammal in need, comprising administering a therapeutically effective amount of a compound of formula I as defined in claim 1, or a pharmaceutically acceptable salt or prodrug thereof, in combination with a pharmaceutically acceptable carrier.
